(19) United States
(12) Patent Application Publication
(10) Pub. No.: US 2017/0240913 A1
(43) Pub. Date: Aug. 24, 2017

(54) Methods and Materials for Producing Coreless Fruit

(71) Applicant: The New Zealand Institute for Plant and Food Research Limited, Auckland (NZ)

(72) Inventors: Robert James SCHAFFER, Auckland (NZ); Hilary Sara IRELAND, Auckland (NZ); Jia-Long YAO, Auckland (NZ)

(73) Assignee: The New Zealand Institute for Plant and Food Research Limited, Auckland (NZ)

(21) Appl. No.: 15/329,229

(22) PCT Filed: Jul. 31, 2015
(86) PCT No.: PCT/IB2015/055802

§ 371 Date: Jan. 25, 2017

(30) Foreign Application Priority Data
Aug. 1, 2014 (NZ) 628200

(51) Int. Cl. C12N 15/82; C12Q 1/68; C07K 14/415

(52) U.S. Cl. CPC C12N 15/8218; C07K 14/415; C12N 15/8262; C12Q 1/6895; C12Q 2600/13; C12N 2310/531; C12Q 2600/158; C12N 2310/14

(57) ABSTRACT

The invention provides materials and methods for producing coreless fruit, or plants that produce coreless fruit. The invention involves combining reduced expression of AGAMOUS (AG) with parthenocarpy. Parthenocarpy can be induced by hormone treatment, or can be provided by reduced or eliminated expression of PISTILATA (PI) or APETALA3 (AP3). The invention provides methods and materials for producing the plants and coreless fruit by genetic modification (GM) and non-GM means. The invention also provides the plants and coreless fruit.

### TECHNICAL FIELD

The present invention relates to methods and materials for producing coreless fruit.

### BACKGROUND ART

Cores present in many fruit carry seed which, under suitable conditions, can germinate to ultimately produce a new fruit bearing plants. Fruit are typically attractive to animals, and seed ingested with the fruit, may be deposited by animals at distant locations from the original fruit bearing plant, resulting in spread of the fruit plant species.

While a seed bearing core is clearly an evolutionary advantage for many of plants, presence of the core can be an inconvenience to humans. The cores of many fruits are fibrous and tough, and are therefore unpleasant for humans to eat and may be difficult to digest. For these reasons cores are often discarded by those eating fruit, or removed before fruit are further processed and/or incorporated into other food products. Such disposal or removal of cores represents a significant waste of the biomass of the fruit, and adds significantly to the cost of fruit processing.

It is therefore an object of the invention to provide novel methods and compositions for producing coreless fruit, or at least to provide the public with a useful choice.

### SUMMARY OF THE INVENTION

Methods

Reducing or Eliminating AG

In one aspect the invention provides a method for producing a coreless fruit, the method comprising reducing, or eliminating, expression of at least one AGAMOUS (AG) protein in a plant.

In a further aspect the invention provides a method for producing a plant that produces at least one coreless fruit, the method comprising reducing or eliminating expression of an AGAMOUS (AG) protein in the plant.

Method Including the Step of Inducing Parthenocarpy

In one embodiment the method includes the additional step of inducing parthenocarpy in the plant.

Therefore in one aspect the invention provides a method for producing a coreless fruit, the method comprising the steps:
a) reducing, or eliminating, expression of at least one AGAMOUS (AG) protein in a plant, and
b) inducing parthenocarpy in the plant.

In a further aspect the invention provides a method for producing a plant that produces at least one coreless fruit, the method comprising the steps:
a) reducing, or eliminating, expression of at least one AGAMOUS (AG) protein in a plant, and
b) inducing parthenocarpy in the plant.

Reducing or Eliminating AG in Parthenocarpic Plant

In one embodiment the plant in which expression of at least one AGAMOUS (AG) protein is reduced or eliminated is a parthenocarpic plant.

Methods for Inducing Parthenocarpy

Pathenocarpy be induced by any means.

In one embodiment parthenocarpy is induced by application of plant hormones to flowers of the plant.

In a further embodiment parthenocarpy is induced manipulating expression of genes controlling fruit set.

In one embodiment parthenocarpy is induced manipulating the expression of at least one PISTILSTA (PI) gene or protein.

In one embodiment parthenocarpy is induced reducing or eliminating expression of at least one PISTILSTA (PI) gene or protein.

In one embodiment parthenocarpy is induced manipulating the expression of at least one APETALA3 (AP3) gene or protein.

In one embodiment parthenocarpy is induced reducing or eliminating expression of at least one APETALA3 (AP3) gene or protein.

Mutant Parthenocarpic Plants

In one embodiment the parthenocarpic plant is a mutant plant with reduced or eliminated expression of at least one PISTILSTA (PI) gene or protein.

In a further embodiment the parthenocarpic plant is a mutant plant with reduced, or eliminated, expression of at least one APETALA3 (AP3) gene or protein.

The mutant plant may be a naturally occurring mutant plant. Alternatively the mutant may be an induced mutant.

Reducing or Eliminating AG and PI

In one aspect the invention provides a method for producing a coreless fruit, the method comprising reducing, or eliminating, expression of at least one AGAMOUS (AG) protein and at least one PISTILATA (PI) protein in a plant.

In a further aspect the invention provides a method for producing a plant that produces at least one coreless fruit, the method comprising reducing or eliminating expression of an AGAMOUS (AG) protein and at least one PISTILATA (PI) protein in the plant.

In one embodiment the reducing or eliminating expression of the at least one PISTILATA (PI) protein induces parthenocarpy.

Reducing or Eliminating AG and AP3

In one aspect the invention provides a method for producing a coreless fruit, the method comprising reducing, or eliminating, expression of at least one AGAMOUS (AG) protein and at least one APETALA3 (AP3) protein in a plant.

In a further aspect the invention provides a method for producing a plant that produces at least one coreless fruit, the method comprising reducing or eliminating expression of an AGAMOUS (AG) protein and at least one APETALA3 (AP3) protein in the plant.

In one embodiment the reducing or eliminating expression of the at least one APETALA3 (AP3) protein induces parthenocarpy.

Non-GM Selection Method for Reduced or Eliminated AGAMOUS (AG)

In a further aspect the invention provides a method for identifying a plant with a genotype indicative of producing, or being useful for producing, at least one coreless fruit, the method comprising testing a plant for at least one of:
a) reduced, or eliminated, expression of at least one AGAMOUS (AG) protein,
b) reduced, or eliminated, expression of at least one polynucleotide encoding an AGAMOUS (AG) protein,
c) presence of a marker associated with reduced expression of at least one AGAMOUS (AG) protein, and
d) presence of a marker associated with reduced expression of at least one polynucleotide encoding an AGAMOUS (AG) protein.

In one embodiment presence of any of a) to d) indicates that the plant will produce, or be useful for producing, at least one coreless fruit.

In a further embodiment the plant identified is a mutant plant with reduced or eliminated expression of an AGAMOUS (AG) gene or protein.

The mutant plant may be a naturally occurring mutant plant. Alternatively the mutant may be an induced mutant.

Non-GM Selection Method for Reduced or Eliminated PISTILATA (PI)

In a further aspect the invention provides a method for identifying a plant with a genotype indicative of producing, or being useful for producing, at least one coreless fruit, the method comprising testing a plant for at least one of:
a) reduced, or eliminated, expression of at least one PISTILATA (PI) protein,
b) reduced, or eliminated, expression of at least one polynucleotide encoding an PISTILATA (PI) protein,
c) presence of a marker associated with reduced expression of at least one PISTILATA (PI) protein, and
d) presence of a marker associated with reduced expression of at least one polynucleotide encoding a PISTILATA (PI) protein.

In one embodiment presence of any of a) to d) indicates that the plant will produce, or be useful for producing, at least one coreless fruit.

In a further embodiment the plant identified is a mutant plant with reduced or eliminated expression of a PISTILATA (PI) gene or protein.

The mutant plant may be a naturally occurring mutant plant. Alternatively the mutant may be an induced mutant.

Non-GM Selection Method for Reduced or Eliminated APETALA3 (AP3)

In a further aspect the invention provides a method for identifying a plant with a genotype indicative of producing, or being useful for producing, at least one coreless fruit, the method comprising testing a plant for at least one of:
a) reduced, or eliminated, expression of at least one APETALA3 (AP3) protein,
b) reduced, or eliminated, expression of at least one polynucleotide encoding an APETALA3 (AP3) protein,
c) presence of a marker associated with reduced expression of at least one APETALA3 (AP3) protein, and
d) presence of a marker associated with reduced expression of at least one polynucleotide encoding a APETALA3 (AP3) protein.

In one embodiment presence of any of a) to d) indicates that the plant will produce, or be useful for producing, at least one coreless fruit.

In a further embodiment the plant identified is a mutant plant with reduced or eliminated expression of an APETALA3 (AP3) gene or protein.

The mutant plant may be a naturally occurring mutant plant. Alternatively the mutant may be an induced mutant.

Methods for Breeding Plants with Coreless Fruit

In a further aspect the invention provides a method for producing a plant that produces at least one coreless fruit, the method comprising crossing one of:
a) a plant of the invention,
b) a plant produced by a method of the invention, and
c) a plant selected by a method of the invention
d) a mutant plant with reduced, or eliminated, expression of one of one of AGAMOUS (AG), PISTILATA (PI), and APETALA3 (AP3)

with another plant, wherein the off-spring produced by the crossing is a plant that produces at least one coreless fruit.

In one embodiment the plant of a), b, c) or d) is a plant with reduced, or eliminated, expression of at least one AGAMOUS (AG) protein. Preferably in this embodiment the another plant is one of:
i) a parthenogenic plant,
ii) a plant with reduced or eliminated expression of at least one PISTILATA (PI) protein,
iii) a plant with reduced or eliminated expression of at least one APETALA3 (AP3) protein

Preferably the plant in i), ii) or iii) is produced or selected by a method of the invention. Alternatively the plant in i), ii) or iii) may be a naturally occurring mutant with reduced or eliminated expression of PISTILATA (PI), and APETALA3 (AP3).

In one embodiment the plant of a), b, or c) is a plant with reduced, or eliminated, expression of at least one PISTILATA (PI) protein. In a further embodiment the plant of a), b, or c) is a plant with reduced, or eliminated, expression of at least one APETALA3 (AP3) protein. Preferably in this embodiment the another plant is a plant with reduced or eliminated expression of at least one AGAMOUS (AG) protein. Preferably the another plant is produced or selected by a method of the invention.

Non-GM Selection Method Including Selecting for Parthenocarpy

In one embodiment the method for identifying a plant with a genotype indicative of producing at least one coreless fruit includes the additional step of identifying a marker of parthenocarpy in the plant.

Method of Producing Coreless Fruit Using Selected Plant

In a further aspect the invention provides a method for producing a coreless fruit, the method comprising cultivating a plant identified by a method of the invention. In one embodiment the method includes the additional step of inducing parthenocarpy in the plant.

In a preferred embodiment the plant produces coreless fruit as a result of the identified plant having reduced or eliminated expression of at least one AGAMOUS (AG) protein.

In a further preferred embodiment the plant produces coreless fruit as a result of the identified plant having reduced, or eliminated expression, of at least one AGAMOUS (AG) protein, and having induced parthenocarpy.

In a further embodiment the plant is produces coreless fruit as a result of the identified plant having reduced, or eliminated expression, of at least one AGAMOUS (AG) protein, and having reduced, or eliminated expression, of one of PISTILATA (PI), and APETALA3 (AP3).

A method of producing a coreless fruit the method comprising cultivating a plant with reduced, or eliminated, expression of at least one of:
a) at least one AGAMOUS (AG) protein, and
b) at least one of:
   i) at least one PISTILATA (PI) protein, and
   ii) at least one APETALA3 (AP3) protein.

Preferably the plant has reduced, or eliminated, expression of both:
a) at least one AGAMOUS (AG) protein, and
b) at least one of:
   i) at least one PISTILATA (PI) protein, and
   ii) at least one APETALA3 (AP3) protein.

Products

Coreless Fruit

In a further aspect the invention provides a coreless fruit produced by a method of the invention.

In a further aspect the invention provides a coreless fruit with reduced or eliminated expression of at least one AGAMOUS (AG) protein

In one embodiment the fruit also has reduced or eliminated expression of at least one PISTILATA (PI) protein.

In a further embodiment the fruit also has reduced or eliminated expression of at least one APETALA3 (AP3) protein.

In a further embodiment the the invention provides a coreless fruit with reduced or eliminated expression of:
a) at least one AGAMOUS (AG) protein, and
b) at least one of:
   i) at least one PISTILATA (PI) protein, and
   ii) at least one APETALA3 (AP3) protein.

Plant that Produces Coreless Fruit

In a further aspect the invention provides a plant, which produces at least one coreless fruit, produced by a method of the invention.

In a further aspect the invention provides a plant, which produces at least one coreless fruit, wherein the plant has reduced or eliminated expression of at least one AGAMOUS (AG) protein.

In one embodiment the fruit also has reduced or eliminated expression of at least one PISTILATA (PI) protein.

In a further embodiment the fruit also has reduced or eliminated expression of at least one APETALA3 (AP3) protein.

In a further embodiment the plant comprises a construct of the invention.

In one embodiment the plant is also parthenocarpic.

In a further embodiment the invention provides a plant, which produces at least one coreless fruit, wherein the plant has reduced or eliminated expression of:
a) at least one AGAMOUS (AG) protein, and
b) at least one of:
   i) at least one PISTILATA (PI) protein, and
   ii) at least one APETALA3 (AP3) protein.

Construct (for Reducing or Eliminating Expression of an AGAMOUS (AG) Protein in a Plant)

In a further aspect the invention provides a construct for reducing the expression of an AGAMOUS (AG) protein in a plant.

In one embodiment the construct is contains a promoter sequence operably linked to at least part of an AGAMOUS (AG) gene, wherein the part of the gene is in an antisense orientation relative to the promoter sequence.

Preferably the part of the gene is at least 21 nucleotides in length.

In one embodiment the construct is an antisense construct.

In a further embodiment the construct is an RNA interference (RNAi) construct.

Construct (for Reducing or Eliminating Expression of an PISTILATA (PI) Protein in a Plant)

In a further aspect the invention provides a construct for reducing the expression of a PISTILATA (PI) protein in a plant.

In one embodiment the construct is contains a promoter sequence operably linked to at least part of an PISTILATA (PI), wherein the part of the gene is in an antisense orientation relative to the promoter sequence.

Preferably the part of the gene is at least 21 nucleotides in length.

In one embodiment the construct is an antisense construct.

In a further embodiment the construct is an RNA interference (RNAi) construct.

Construct (for Reducing or Eliminating Expression of an APETALA3 (AP3) Protein in a Plant)

In a further aspect the invention provides a construct for reducing the expression of a APETALA3 (AP3) protein in a plant.

In one embodiment the construct is contains a promoter sequence operably linked to at least part of an APETALA3 (AP3), wherein the part of the gene is in an antisense orientation relative to the promoter sequence.

Preferably the part of the gene is at least 21 nucleotides in length.

In one embodiment the construct is an antisense construct.

In a further embodiment the construct is an RNA interference (RNAi) construct.

Plant/Fruit

The plant may be from any species that, without application of the method of the invention, produces fruit with a core.

In one embodiment the plant is from a species that produces accessory fruit.

Preferred plants that produce accessory fruit include apple and pear plants.

A preferred apple genus is Malus.

Preferred apple species include: Malus angustifolia, Malus asiatica, Malus baccata, Malus coronaria, Malus doumeri, Malus florentina, Malus floribunda, Malus fusca, Malus halliana, Malus honanensis, Malus hupehensis, Malus ioensis, Malus kansuensis, Malus mandshurica, Malus micromalus, Malus niedzwetzkyana, Malus ombrophilia, Malus orientalis, Malus prattii, Malus prunifolia, Malus pumila, Malus sargentii, Malus sieboldii, Malus sieversii, Malus sylvestris, Malus toringoides, Malus transitoria, Malus trilobata, Malus tschonoskii, Malus×domestica, Malus×domestica×Malus sieversii, Malus×domestica×Pyrus communis, Malus xiaojinensis, and Malus yunnanensis.

A particularly preferred apple species is Malus×domestica.

A preferred pear genus is Pyrus.

Preferred pear species include: Pyrus calleryana, Pyrus caucasica, Pyrus communis, Pyrus elaeagrifolia, Pyrus hybrid cultivar, Pyrus pyrifolia, Pyrus salicifolia, Pyrus ussuriensis and Pyrus×bretschneideri.

A particularly preferred pear species is Pyrus communis, and Asian pear Pyrus×bretschneideri

Other preferred plants include quince, loquat, and hawthorn.

A preferred quince genus is Chaenomeles

Preferred quince species include: Chaenomeles cathayensis and Chaenomeles speciosa.

A particularly preferred quince species is Chaenomeles speciosa.

A preferred loquat genus is Eriobotrya

Preferred loquat species include: Eriobotrya japonica and Eriobotrya japonica

A particularly preferred loquat species is Eriobotrya japonica

A preferred hawthorn genus is Crataegus.

Preferred hawthorn species include: Crataegus azarolus, Crataegus columbiana, Crataegus crus-galli, Crataegus curvisepala, Crataegus laevigata, Crataegus mollis, Crataegus monogyna, Crataegus nigra, Crataegus rivularis, and Crataegus sinaic.

Plant Parts, Propagules and Progeny

In a further embodiment the invention provides a part, progeny, or propagule of a plant of the invention.

Preferably the part, progeny, or propagule has reduced or eliminated expression of at least one AGAMOUS (AG) protein.

In one embodiment the part, progeny, propagule has reduced or eliminated expression of at least one PISTILATA (PI) protein.

In a further embodiment the part, progeny, propagule has reduced or eliminated expression of at least one APETALA3 (AP3) protein.

Preferably the part, progeny, propagule comprises a construct of the invention.

The term “part” of a plant refers to any part of the plant. The term “part” preferably includes any one of the following: tissue, organ, fruit, and seed.

The term “propagule” of a plant preferably includes any part of a plant that can be used to regenerate a new plant. Preferably the term “propagule” includes seeds and cuttings.

The term “progeny” includes any subsequent generation of plant. The progeny may be produced as a result of sexual crossing with another plant. The progeny plant may also be asexually produced.

### DETAILED DESCRIPTION OF THE INVENTION

The invention provides materials and methods for producing coreless fruit, or plants that produce coreless fruit. The invention involves combining reduced expression of AGAMOUS (AG) with parthenocarpy. Parthenocarpy can be induced by hormone treatment, or can be provided by reduced or eliminated expression of PISTILATA (PI) or APETALA3 (AP3). The invention provides methods and materials for producing the plants and coreless fruit by genetic modification (GM) and non-GM means. The invention also provides the plants and coreless fruit.

Those skilled in the art will appreciate that plants with reduced or eliminated expression of AGAMOUS (AG), and reduced or eliminated expression of PISTILATA (PI) or APETALA3 (AP3), can be produced in many different ways. Plants with reduced expression of one or more of the genes can be produced by genetic modification (GM) approaches, or can be selected, or provided as naturally occurring mutants. Crossing of GM or non-GM plants can be used to generate plants with the desired combination of reduced or eliminated gene expression. Similarly a GM approach can be used to reduce expression of one of the genes in a naturally occurring or selected mutant that has reduced expression of the other required gene.

Regardless of how they are produced, the invention preferably encompasses, any coreless fruit, or plant that produces coreless fruit, wherein the plant or coreless fruit has reduced or eliminated expression of AGAMOUS (AG), and reduced or eliminated expression of PISTILATA (PI) or APETALA3 (AP3). The invention also encompasses the methods for producing such plants and coreless fruit as described herein.

Definitions

Core

The term “core” of a fruit refers to the fibrous tissue in the centre of the apples containing locular cavities, and seeds.

Coreless

The term “coreless” as used herein means lacking a core. A “coreless” fruit according to the invention therefore preferably also lacks seeds. A “coreless” fruit according to the invention therefore preferable also lacks locular cavities.

Accessory Fruit

Unlike true fruit which are derived from ovary tissue, accessory fruits are derived from other floral or receptacle tissue.

In the case of pipfruit, such as apples and pears the fruit flesh is derived from the hypanthium which is a tube of sepal, petal and stamen tissue surrounding the carpel.

Hypanthium

The hypanthium tissue surrounds the carpel which forms the core of the fruit.

Floral Organ Identity A, B and C Function Genes

All flowers have whorls of floral organs defined as sepals, petals, stamens and carpels. The production each of these organ types is determined by a set of MADS box transcription factors, commonly described as A, B and C function genes. A function genes such as APETELA1 control sepal and petal determination. B function genes such as PISTILATA (PI) and APETALA3 (AP3), control petal and stamen determination. C function gene such as AGAMOUS (AG) control stamen and carpel determination.

All AG, PI, and AP3 proteins have two conserved motifs, the MADS domain for DNA binding and the K domain for protein-protein interaction, as illustrated in FIG. 9.

AGAMOUS (AG) Protein

AGAMOUS (AG) proteins, and the genes encoding them, are well known to those skilled in the art.

For example The AGAMOUS cluster in model plant Arabidopsis thaliana consists of 4 genes known as AG, SEEDSTICK (STK), SHATTERPROOF (SHP) 1 and 2.

The AGAMOUS (AG) protein according to the invention may be any AGAMOUS protein.

In one embodiment the AGAMOUS protein comprises at least one of a MADS domain and a K domain as illustrated in FIG. 9. Preferably the AGAMOUS protein comprises both a MADS domain and a K domain as illustrated in FIG. 9.

In a further embodiment, the AGAMOUS protein has at least 70% sequence identity to any one of the AGAMOUS proteins referred to in Table 1 below (and presented in the sequence listing).

In a further embodiment the AGAMOUS protein is one of the AGAMOUS proteins referred to in Table 1 below (and presented in the sequence listing).

In a preferred embodiment the AGAMOUS protein has at least 70% sequence identity to the sequence of SEQ ID NO: 1.

In a preferred embodiment the AGAMOUS protein has the sequence of SEQ ID NO: 1.

Polynucleotide Encoding an AGAMOUS (AG) Protein

In one embodiment, the sequence encoding the AGAMOUS protein has at least 70% sequence identity to any one of the AGAMOUS polynucleotides referred to in Table 1 below (and presented in the sequence listing).

In a further embodiment the sequence encoding the AGAMOUS protein is one of the AGAMOUS polynucleotides referred to in Table 1 below (and presented in the sequence listing).

In a preferred embodiment the sequence encoding the AGAMOUS protein has at least 70% sequence identity to the sequence of SEQ ID NO: 4.

In a preferred embodiment the sequence encoding the AGAMOUS protein has the sequence of SEQ ID NO: 4.

TABLE 1
AGAMOUS sequences

| SEQ | | | | |
|---|---|---|---|---|
| ID | Sequence | Common | | |
| NO: | type | name | Species | Reference |
| 1 | Polypeptide | Apple | Malus × | MdAG |
| | | | domestica | |
| 2 | Polypeptide | Pear | Pyrus | PbAG, Pbr039503.1 |
| | | | bretschneideri | |
| 3 | Polypeptide | Pear | Pyrus | PcAG, PCP031198 |
| | | | communis | |
| 4 | Polynucleotide | Apple | Malus × | MdAG |
| | | | domestica | |
| 5 | Polynucleotide | Pear | Pyrus | PbAG, Pbr039503.1 |
| | | | bretschneideri | |
| 6 | Polynucleotide | Pear | Pyrus | PcAG, PCP031198 |
| | | | communis | |
| 30 | Polypeptide | Apple | Malus × | MADS15 |
| | | | domestica | |
| 31 | Polynucleotide | Apple | Malus × | MADS15 |
| | | | domestica | |

AGAMOUS (AG) Gene

The AGAMOUS (AG) gene according to the invention may be any AGAMOUS (AG) gene.

Preferably the AGAMOUS (AG) gene encodes an AGAMOUS (AG) protein as herein defined.

Gene

A term “gene” as used herein may be the target for reducing, or eliminating, expression of an AGAMOUS (AG), PISTILATA (PI) or APETALA3 (AP3) protein or polynucleotide.

The term gene include the sequence encoding the protein, which may be separate exons, any regulatory sequences (including promoter and terminator sequences) 5′ and 3′ untranslated sequence, and introns.

It is known by those skilled in the art that any of such features of the gene may be targeted in silencing approaches such as antisense, sense suppression and RNA interference (RNAi).

Methods for Reducing, or Eliminating, Expression of Proteins/Genes

The terms reduced expression, reducing expression and grammatical equivalents thereof means reduced/reducing expression relative to at least one of:
a wild type plant
a non-transformed plant
a plant transformed with a control construct
a non selected plant

A control construct may be for example an empty vector construct.

Methods for reducing or eliminating expression of proteins/polynucleotides/genes are known in the art, and are described herein.

Pathenocarpy

Pathenocarpy is the production of fruit in the absence of pollination.

Methods for Inducing Parthenocarpy

Methods for inducing parthenocarpy in plants have been reported in the art. Pathenocarpy can be induced with hormone treatment or genetically with the modulation of certain genes detailed in (Sotelo-Silveira et al., 2014). In apples extensive work was done to induce parthenocarpy, only the triple combination of GA3, SD8339, and 2-NAA, rather than single or paired application, resulted in parthenocarpy in Cox's Orange Pippin (Kotob & Schwabe 1971) and GA4+7 alone induced parthenocarpy in frost-damaged Bramley's Seedling and cytokinin SD8339 had no additional benefits; GA3 was not effective. This said, Bramley's Seedling is triploid and partially self-fertile so may be an unusual case (Modlibowska 1972).

Methods for inducing parthenocarpy according to the invention include application of plant hormones to flowers of the plant concerned.

In one embodiment parthenocarpy is induced by applying at least one of:
a) an auxin
b) a cytokinin
c) a giberellin

Preferably at least two, more preferably all three of a), b) and c) are applied. When two are applied, preferably the two are a) and c).

Preferred auxins include: IAA, NAA, 2,4-D and IBA.

A preferred auxin is IAA

Preferred cytokinins include: BAP, CPPU, Zeatin, TDZ and kinetin.

A preferred cytokinin is BAP

Preferred giberellins include: GA1, GA3, GA4 and GA7.

A preferred giberellin is GA4

Preferably the auxin concentration is in the range 0.01 to 100 ppm, more preferably 0.1 to 10 ppm, more preferably 0.2 to 5 ppm, more preferably 0.5 to 2 ppm, more preferably about 1 ppm, more preferably 1 ppm.

Preferably the cytokinin concentration is in the range 1 to 10,000 ppm, more preferably 10 to 1000 ppm, more preferably 20 to 500 ppm, more preferably 50 to 200 ppm, more preferably about 100 ppm, more preferably 100 ppm.

Preferably the giberellin concentration is in the range 3 to 30,000 ppm, more preferably 30 to 3000 ppm, more preferably 60 to 1500 ppm, more preferably 150 to 600 ppm, more preferably 200 to 400 ppm, more preferably 250 to 350, more preferably about 300 ppm, more preferably 300 ppm.

Preferably flowers are treated before full bloom.

Preferably treatment commences on, or earlier than: one day after full bloom (+1 DAFB), more preferably on the day of full bloom, more preferably at least 1 day before full bloom (−1 DAFB), more preferably at least 2 days before full bloom (−2 DAFB), more preferably at least 3 days before full bloom (−3 DAFB), more preferably at least 4 days before full bloom (−4 DAFB), more preferably at least 5 days before full bloom (−5 DAFB), more preferably at least 6 days before full bloom (−6 DAFB), more preferably at least 7 days before full bloom (−7 DAFB).

Preferably flowers are treated at least once, more preferably at least twice, more preferably at least three times, more preferably at least four times.

Preferably treatments are at intervals of at least one day, preferably at least 2 days, preferably at least 3 days, preferably at least 4 days.

In one embodiment treatments are at −7, −4 and +1 DAFB.

DAFB means days after flower bloom.

In one embodiment flowers with partial ovules are treated with auxin and giberellin only.

In a further embodiment with no ovule tissue are treated with auxin, cytokinin and giberellin.

Inducing Parthenocarpy by Manipulating Gene Expression

Other methods for inducing parthenocarpy include manipulating the expression of target genes.

For example this has been achieved in apple through eliminating expression of a PISTILATA (PI) protein (Yao et al., “Parthenocarpic apple fruit production conferred by transposon insertion mutations in a MADS-box transcription factor.” Proceedings of the National Academy of Sciences 98.3 (2001): 1306-1311)

In one embodiment the method for inducing parthenocarpy comprises reducing, or eliminating expression of a PISTILATA (PI) protein.

PISTILATA (PI) Protein

PISTILATA (PI) proteins, and the genes encoding them, are well known to those skilled in the art.

Knocking-out PISTILATA (PI) gene in apple produces flowers with two whorls of sepals and two whorls of carpels, but no petals or stamens. These flowers can develop parthenocarpic fruit. This may be due to the enhancement of sepal development helping fruit set without pollination.

The PISTILATA (PI) protein according to the invention may be any PISTILATA protein.

In one embodiment the PISTILATA protein comprises at least one of a MADS domain and a K domain as illustrated in FIG. 9. Preferably the PISTILATA protein comprises both a MADS domain and a K domain as illustrated in FIG. 9.

In a further embodiment, the PISTILATA protein has at least 70% sequence identity to any one of the PISTILATA proteins referred to in Table 2 below (and presented in the sequence listing).

In a further embodiment the PISTILATA protein is one of the PISTILATA proteins referred to in Table 1 below (and presented in the sequence listing).

In a preferred embodiment the PISTILATA protein has at least 70% sequence identity to the sequence of SEQ ID NO: 7.

In a preferred embodiment the PISTILATA protein has the sequence of SEQ ID NO: 7.

Polynucleotide Encoding a PISTILATA (PI) Protein

In one embodiment, the sequence encoding the PISTILATA protein has at least 70% sequence identity to any one of the PISTILATA polynucleotides referred to in Table 1 below (and presented in the sequence listing).

In a further embodiment the sequence encoding the PISTILATA protein is one of the PISTILATA polynucleotides referred to in Table 1 below (and presented in the sequence listing).

In a preferred embodiment the sequence encoding the PISTILATA protein has at least 70% sequence identity to the sequence of SEQ ID NO: 10.

In a preferred embodiment the sequence encoding the PISTILATA protein has the sequence of SEQ ID NO: 10.

TABLE 2
PISTILATA sequences

| SEQ | | | | |
|---|---|---|---|---|
| ID | | Sequence | Common | |
| NO: | type | name | Species | Reference |
| 7 | Polypep- | Apple | Malus × | MdPI, GenBank: |
| | tide | | domestica | AJ291490 |
| 8 | Polypep- | Pear | Pyrus | PcPI, PCP018702 |
| | tide | | communis | scaffold01412 3716 |
| | | | | 6783 + 1 654 |
| | | | | oldname = |
| | | | | AUG2gene00029316 |
| 9 | Polypep- | Pear | Pyrus × | PbPI, Pbr035294.1 |
| | tide | | bretschneideri | |
| 10 | Polynucleo- | Apple | Malus × | MdPI, GenBank: |
| | tide | | domestica | AJ291490 |
| 11 | Polynucleo- | Pear | Pyrus | PcPI, PCP018702 |
| | tide | | communis | scaffold01412 3716 |
| | | | | 6783 + 1 654 |
| | | | | oldname = |
| | | | | AUG2gene00029316 |
| 12 | Polynucleo- | Pear | Pyrus × | PbPI, Pbr035294.1 |
| | tide | | bretschneideri | |

PISTILATA (PI) Gene

The PISTILATA (PI) gene according to the invention may be any PISTILATA (PI) gene.

Preferably the PISTILATA (PI) gene encodes a PISTILATA (PI) protein as herein defined.

APETALA3 (AP3)

APETALA3 (AP3) is known to form a heterodimer with PISTILATA (PI) The proteins encoded by AP3 and PI are stable and functional in the cell only as heterodimers (Winter, K. U. et al. 2002, Evolution of class B floral homeotic proteins: Obligate heterodimerization originated from homodimerization. Molecular Biology and Evolution 19, 587-596). Further more, knocking-out AP3 gives the same phenotype as knock-out PISTILATA (PI) (Weigel, D. & Meyerowitz, E. M. 1994, The ABCs of floral homeotic genes. Cell 78, 203-209). Therefore parthenocarpy may also be induced by reducing, or eliminating expression of an APETALA3 (AP3) protein.

In one embodiment the method for inducing parthenocarpy comprises reducing, or eliminating expression of an APETALA3 (AP3) protein.

APETALA3 (AP3) Protein

APETALA3 (AP3) proteins, and the genes encoding them, are well known to those skilled in the art.

The APETALA3 (AP3) protein according to the invention may be any APETALA3 (AP3) protein.

In one embodiment the APETALA3 (AP3) protein comprises at least one of a MADS domain and a K domain as illustrated in FIG. 9. Preferably the APETALA3 (AP3) protein comprises both a MADS domain and a K domain as illustrated in FIG. 9.

In a further embodiment, the APETALA3 (AP3) protein has at least 70% sequence identity to any one of the APETALA3 (AP3) proteins referred to in Table 3 below (and presented in the sequence listing).

In a further embodiment the APETALA3 (AP3) protein is one of the APETALA3 (AP3) proteins referred to in Table 3 below (and presented in the sequence listing).

In a preferred embodiment the APETALA3 (AP3) protein has at least 70% sequence identity to the sequence of SEQ ID NO: 13 or 14.

In a preferred embodiment the APETALA3 (AP3) protein has the sequence of SEQ ID NO: 13 or 14.

Polynucleotide Encoding a APETALA3 (AP3) Protein

In one embodiment, the sequence encoding the APETALA3 (AP3) protein has at least 70% sequence identity to any one of the APETALA3 (AP3) polynucleotides referred to in Table 3 below (and presented in the sequence listing).

In a further embodiment the sequence encoding the APETALA3 (AP3) protein is one of the APETALA3 (AP3) polynucleotides referred to in Table 3 below (and presented in the sequence listing).

In a preferred embodiment the sequence encoding the APETALA3 (AP3) protein has at least 70% sequence identity to the sequence of SEQ ID NO: 19 or 20.

In a preferred embodiment the sequence encoding the APETALA3 (AP3) protein has the sequence of SEQ ID NO: 19 or 20.

TABLE 3
APETALA3 (AP3) sequences

| | Sequence | Common | | |
|---|---|---|---|---|
| | type | name | Species | Reference |
| 13 | Polypep- | Apple | Malus × | MdMT6, GenBank: |
| | tide | | domestica | AB081093 |
| 14 | Polypep- | Apple | Malus × | MdMADS13 GenBank; |
| | tide | | domestica | AJ251116 |
| 15 | Polypep- | Pear | Pyrus | Pbr022146.1 |
| | tide | | bretschneideri | |
| 16 | Polypep- | Pear | Pyrus | Pbr040541.1 |
| | tide | | bretschneideri | |
| 17 | Polypep- | Pear | Pyrus | PCP014552 |
| | tide | | communis | scaffold00010 |
| | | | | 589169 591493 + 1 714 |
| | | | | oldname = |
| | | | | AUG2gene00023123.1 |
| 18 | Polypep- | Pear | Pyrus | PCP002673 |
| | tide | | communis | scaffold 01202 |
| | | | | 29036 31550 + 1 705 |
| | | | | oldname = |
| | | | | AUG2gene00003856.1 |
| 19 | Polynucleo- | Apple | Malus × | MdMT6, GenBank: |
| | tide | | domestica | AB081093 |
| 20 | Polynucleo- | Apple | Malus × | MdMADS13 GenBank; |
| | tide | | domestica | AJ251116 |
| 21 | Polynucleo- | Pear | Pyrus | Pbr022146.1 |
| | tide | | bretschneideri | |
| 22 | Polynucleo- | Pear | Pyrus | Pbr040541.1 |
| | tide | | bretschneideri | |
| 23 | Polynucleo- | Pear | Pyrus | PCP014552 |
| | tide | | communis | scaffold00010 |
| | | | | 589169 591493 + 1 714 |
| | | | | oldname = |
| | | | | AUG2gene00023123.1 |
| 24 | Polynucleo- | Pear | Pyrus | PCP002673 |
| | tide | | communis | scaffold01202 |
| | | | | 29036 31550 + 1 705 |
| | | | | oldname = |
| | | | | AUG2gene00003856.1 |

APETALA3 (AP3) Gene

The APETALA3 (AP3) gene according to the invention may be any APETALA3 (AP3) gene.

Preferably the APETALA3 (AP3) gene encodes a APETALA3 (AP3) protein as herein defined.

Marker Assisted Selection

Marker assisted selection (MAS) is an approach that is often used to identify plants that possess a particular trait using a genetic marker, or markers, associated with that trait. MAS may allow breeders to identify and select plants at a young age and is particularly valuable for hard to measure traits. The best markers for MAS are the causal mutations, but where these are not available, a marker that is in strong linkage disequilibrium with the causal mutation can also be used. Such information can be used to accelerate genetic gain, or reduce trait measurement costs, and thereby has utility in commercial breeding programs.

Markers

Markers for use in the methods of the invention may include nucleic acid markers, such as single nucleotide polymorphisms (SNPs), simple sequence repeats (SSRs or microsatellites), insertions, substitutions, indels and deletions.

Preferably the marker is in linkage disequilibrium (LD) with the trait.

Preferably the marker is in LD with the trait at a D′ value of at least 0.1, more preferably at least 0.2, more preferably at least 0.3, more preferably at least 0.4, more preferably at least 0.5.

Preferably the marker is in LD with the trait at a R² value of at least 0.05, more preferably at least 0.075, more preferably at least 0.1, more preferably at least 0.2, more preferably at least 0.3, more preferably at least 0.4, more preferably at least 0.5.

The term “linkage disequilibrium” or LD as used herein, refers to a derived statistical measure of the strength of the association or co-occurrence of two independent genetic markers. Various statistical methods can be used to summarize linkage disequilibrium (LD) between two markers but in practice only two, termed D′ and R², are widely used.

Marker linked, and or in LD, with the trait may be of any type including but not limited to, SNPs, substitutions, insertions, deletions, indels, simple sequence repeats (SSRs).

Methods for marker assisted selection are well known to those skilled in the art.

Mutant Parthenocarpic Plants

Mutant parthenocarpic plants preferably have reduced expression of at least one of PISTILATA (PI), and APETALA3 (AP3).

In one embodiment the parthenocarpic plants have reduced expression of PISTILATA (PI). An example of such parthenocarpic plants is the ‘Rae Ime’ apple mutant which has an insertion in an intron in the PI gene, and does not express the apple MdPI gene. ‘Spencer Seedless’ and ‘Wellington Bloomless’ apple mutants also have the same phenotype and a similar insertion (Yao et al. 2001).

Plants with similar phenotype can be selected for whole genome sequencing to identify mutations in the AP3 or PI genes, and for q-RT-PCR analysis to confirm the reduced, or eliminated, expression of the AP3 or PI. Alternatively plants can be screened for reduced, or eliminated, expression of the AP3 or PI first.

Mutant AGAMOUS Plants

Mutant AGAMOUS plants preferably have reduced expression of AGAMOUS (AG).

Plants can be identified which show a similar phenotype to the AG suppression transgenic plants, described in Example 1.

Plants with such a phenotype can be selected for whole genome sequencing to identify mutations in the AG genes, and for q-RT-PCR analysis to confirm the reduced, or eliminated, expression of the AG gene. Alternatively plants can be screened for reduced, or eliminated, expression of the AG gene first.

Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

Preferably the term “polynucleotide” includes both the specified sequence and its compliment.

A “fragment” of a polynucleotide sequence provided herein is a subsequence of contiguous nucleotides, e.g., a sequence that is at least 15 nucleotides in length. The fragments of the invention comprise 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide of the invention. Fragments of polynucleotides for use in silence, in particular for RNA interference (RNAi) approaches are preferably at least 21 nucleotides in length.

The term “primer” refers to a short polynucleotide, usually having a free 3′OH group that is hybridized to a template and used for priming polymerization of a polynucleotide complementary to the target.

Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides of the present invention may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide. In one embodiment the fragment can perform the same function as the full length polypeptide from which it is derived, or is part of. Preferably the fragment performs a function that is required for the biological activity and/or provides three dimensional structure of the polypeptide.

The term “isolated” as applied to the polynucleotide or polypeptide sequences disclosed herein is used to refer to sequences that are removed from their natural cellular environment. In one embodiment the sequence is separated from its flanking sequences as found in nature. An isolated molecule may be obtained by any method or combination of methods including biochemical, recombinant, and synthetic techniques.

The term “recombinant” refers to a polynucleotide sequence that is synthetically produced or is removed from sequences that surround it in its natural context. The recombinant sequence may be recombined with sequences that are not present in its natural context.

A “recombinant” polypeptide sequence is produced by translation from a “recombinant” polynucleotide sequence.

The term “derived from” with respect to polynucleotides or polypeptides of the invention being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

Variants

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the polypeptides and polynucleotides disclosed herein possess biological activities that are the same or similar to those of the disclosed polypeptides or polypeptides. The term “variant” with reference to polypeptides and polynucleotides encompasses all forms of polypeptides and polynucleotides as defined herein.

Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present invention. Identity is found over a comparison window of at least 20 nucleotide positions, preferably at least 50 nucleotide positions, more preferably at least 100 nucleotide positions, and most preferably over the entire length of a polynucleotide of the invention.

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). In one embodiment the default parameters of bl2seq are utilized. In a further except the default parameters of bl2seq are utilized, except that filtering of low complexity parts should be turned off.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp.276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

A preferred method for calculating polynucleotide % sequence identity is based on aligning sequences to be compared using Clustal X (Jeanmougin et al., 1998, Trends Biochem. Sci. 23, 403-5.)

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp://ftp.ncbi.nih.gov/blast/).

Alternatively, variant polynucleotides of the present invention hybridize to the specified polynucleotide sequences, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency. With respect to polynucleotide molecules greater than about 100 bases in length, typical stringent hybridization conditions are no more than 25 to 30 o C (for example, 10 o C) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0. 41% (G+C−log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65 oC, 6×SSC, 0.2% SDS overnight; followed by two washes of 30 minutes each in 1× SSC, 0.1% SDS at 65 o C and two washes of 30 minutes each in 0.2×SSC, 0.1% SDS at 65 oC.

With respect to polynucleotide molecules having a length less than 100 bases, exemplary stringent hybridization conditions are 5 to 10 o C below Tm. On average, the Tm of a polynucleotide molecule of length less than 100 bp is reduced by approximately (500/oligonucleotide length) o C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10 o C below the Tm.

Variant polynucleotides of the present invention also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [November 2002]) from NCBI (ftp://ftp.ncbi.nih.gov/blast/) via the tblastx algorithm as previously described.

Polypeptide Variants

The term “variant” with reference to polypeptides encompasses naturally occurring, recombinantly and synthetically produced polypeptides. Variant polypeptide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available from NCBI (ftp://ftp.ncbi.nih.gov/blast/). In one embodiment the default parameters of bl2seq are utilized. In a further except the default parameters of bl2seq are utilized, except that filtering of low complexity parts should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

A preferred method for calculating polypeptide % sequence identity is based on aligning sequences to be compared using Clustal X (Jeanmougin et al., 1998, Trends Biochem. Sci. 23, 403-5.)

A variant polypeptide includes a polypeptide wherein the amino acid sequence differs from a polypeptide herein by one or more conservative amino acid substitutions, deletions, additions or insertions which do not affect the biological activity of the peptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagines, glutamine; serine, threonine; lysine, arginine; and phenylalanine, tyrosine.

Non-conservative substitutions will entail exchanging a member of one of these classes for a member of another class.

Analysis of evolved biological sequences has shown that not all sequence changes are equally likely, reflecting at least in part the differences in conservative versus non-conservative substitutions at a biological level. For example, certain amino acid substitutions may occur frequently, whereas others are very rare. Evolutionary changes or substitutions in amino acid residues can be modelled by a scoring matrix also referred to as a substitution matrix. Such matrices are used in bioinformatics analysis to identify relationships between sequences, one example being the BLOSUM62 matrix shown below (Table 4).

TABLE 4
The BLOSUM62 matrix containing all possible substitution scores
[Henikoff and Henikoff, 1992].

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −3 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | −1 | −1 | −2 | −2 | 0 | −3 | −1 | 4 |

The BLOSUM62 matrix shown is used to generate a score for each aligned amino acid pair found at the intersection of the corresponding column and row. For example, the substitution score from a glutamic acid residue (E) to an aspartic acid residue (D) is 2. The diagonal show scores for amino acids which have not changed. Most substitutions changes have a negative score. The matrix contains only whole numbers.

Determination of an appropriate scoring matrix to produce the best alignment for a given set of sequences is believed to be within the skill of in the art. The BLOSUM62 matrix in table 1 is also used as the default matrix in BLAST searches, although not limited thereto.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g. D-amino acids or non-naturally occurring synthetic amino acids, e.g. beta or gamma amino acids and cyclic analogs

Constructs, Vectors and Components Thereof

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

In one embodiment at least one of the promoter and terminator is heterologous with respect to the polynucleotide to be expressed. In one embodiment the promoter is heterologous with respect to the polynucleotide to be expressed. In a further embodiment the terminator is heterologous with respect to the polynucleotide to be expressed. The term “heterologous” means that the sequences, that are heterologous to each other, are not found together in nature. Preferably the sequences are not found operably linked in nature. In one embodiment, the heterologous sequences are found in different species. However, one or more of the heterologous sequences may also be synthetically produced and not found in nature at all.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequence of interest, such as a sequence to be expressed is placed under the control of, and typically connected to another sequence comprising regulatory elements that may include promoters, tissue-specific regulatory elements, temporal regulatory elements, enhancers, repressors and terminators, 5′-UTR sequences, 5′-UTR sequences comprising uORFs, and uORFs.

The term “noncoding region” refers to untranslated sequences that are upstream of the translational start site and downstream of the translational stop site. These sequences are also referred to respectively as the 5′-UTR and the 3′-UTR. These regions include elements required for transcription initiation and termination and for regulation of translation efficiency.

A 5′-UTR sequence is the sequence between the transcription initiation site, and the translation start site.

The 5′-UTR sequence is an mRNA sequence encoded by the genomic DNA. However as used herein the term 5′-UTR sequence includes the genomic sequence encoding the 5′-UTR sequence, and the compliment of that genomic sequence, and the 5′-UTR mRNA sequence.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to cis-regulatory elements upstream of the coding region that regulate gene transcription. Promoters comprise cis-initiator elements which specify the transcription initiation site and conserved boxes such as the TATA box, and motifs that are bound by transcription factors.

A “transgene” is a polynucleotide that is introduced into an organism by transformation. The transgene may be derived from the same species or from a different species as the species of the organism into which the transgene is introduced. The transgenet may also be synthetic and not found in nature in any species.

A “transgenic plant” refers to a plant which contains new genetic material as a result of genetic manipulation or transformation. The new genetic material may be derived from a plant of the same species as the resulting transgenic plant or from a different species, or may be synthetic.

Preferably the “transgenic” is different from any plant found in nature due the the presence of the transgene.

An “inverted repeat” is a sequence that is repeated, where the second half of the repeat is in the complementary strand, e.g.,

(5′)GATCTA . . . TAGATC(3′)
(3′)CTAGAT . . . ATCTAG(5′)

Read-through transcription will produce a transcript that undergoes complementary base-pairing to form a hairpin structure provided that there is a 3-5 bp spacer between the repeated regions.

The terms “to alter expression of” and “altered expression” of a polynucleotide or polypeptide of the invention, are intended to encompass the situation where genomic DNA corresponding to a polynucleotide of the invention is modified thus leading to altered expression of a polynucleotide or polypeptide of the invention. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

Methods for Isolating or Producing Polynucleotides

The polynucleotide molecules of the invention can be isolated by using a variety of techniques known to those of ordinary skill in the art. By way of example, such polypeptides can be isolated through use of the polymerase chain reaction (PCR) described in Mullis et al., Eds. 1994 The Polymerase Chain Reaction, Birkhauser, incorporated herein by reference. The polypeptides of the invention can be amplified using primers, as defined herein, derived from the polynucleotide sequences of the invention.

Further methods for isolating polynucleotides of the invention include use of all, or portions of, the polypeptides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1× Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the invention may be produced by techniques well-known in the art such as restriction endonuclease digestion, oligonucleotide synthesis and PCR amplification.

A partial polynucleotide sequence may be used, in methods well-known in the art to identify the corresponding full length polynucleotide sequence. Such methods include PCR-based methods, 5′RACE (Frohman M A, 1993, Methods Enzymol. 218: 340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilise a sequence identical (or at least highly similar) to that in the plant, for which reduced expression is desired. For these reasons among others, it is desirable to be able to identify and isolate orthologues of a particular gene in several different plant species.

Variants (including orthologues) may be identified by the methods described.

Methods for Identifying Variants

Physical Methods

Variant polypeptides may be identified using PCR-based methods (Mullis et al., Eds. 1994 The Polymerase Chain Reaction, Birkhauser). Typically, the polynucleotide sequence of a primer, useful to amplify variants of polynucleotide molecules of the invention by PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides of the invention (Sambrook et al., Molecular Cloning: A Laboratory Manual, 2nd Ed. Cold Spring Harbor Press, 1987) or by identifying polypeptides from natural sources with the aid of such antibodies.

Computer Based Methods

The variant sequences of the invention, including both polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217))or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

Methods for Isolating Polypeptides

The polypeptides of the invention, including variant polypeptides, may be prepared using peptide synthesis methods well known in the art such as direct peptide synthesis using solid phase techniques (e.g. Stewart et al., 1969, in Solid-Phase Peptide Synthesis, WH Freeman Co, San Francisco Calif., or automated synthesis, for example using an Applied Biosystems 431A Peptide Synthesizer (Foster City, Calif.). Mutated forms of the polypeptides may also be produced during such syntheses.

The polypeptides and variant polypeptides of the invention may also be purified from natural sources using a variety of techniques that are well known in the art (e.g. Deutscher, 1990, Ed, Methods in Enzymology, Vol. 182, Guide to Protein Purification).

Alternatively the polypeptides and variant polypeptides of the invention may be expressed recombinantly in suitable host cells and separated from the cells as discussed below.

Methods for Modifying Sequences

Methods for modifying the sequence of proteins, or the polynucleotide sequences encoding them, are well known to those skilled in the art. The sequence of a protein may be conveniently be modified by altering/modifying the sequence encoding the protein and expressing the modified protein. Approaches such as site-directed mutagenesis may be applied to modify existing polynucleotide sequences. Alternatively restriction endonucleases may be used to excise parts of existing sequences. Altered polynucleotide sequences may also be conveniently synthesised in a modified form.

Methods for Producing Constructs and Vectors

The genetic constructs of the present invention comprise one or more polynucleotide sequences of the invention and/or polynucleotides encoding polypeptides of the invention, and may be useful for transforming, for example, bacterial, fungal, insect, mammalian or plant organisms. The genetic constructs of the invention are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

Methods for Producing Host Cells Comprising Polynucleotides, Constructs or Vectors

The invention provides a host cell which comprises a genetic construct or vector of the invention. Host cells may be derived from, for example, bacterial, fungal, insect, mammalian or plant organisms.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

Methods for Producing Plant Cells and Plants Comprising Constructs and Vectors

The invention further provides plant cells which comprise a genetic construct of the invention, and plant cells modified to alter expression of a polynucleotide or polypeptide of the invention. Plants comprising such cells also form an aspect of the invention.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

Methods for Genetic Manipulation of Plants

A number of plant transformation strategies are available (e.g. Birch, 1997, Ann Rev Plant Phys Plant Mol Biol, 48, 297, Hellens R P, et al (2000) Plant Mol Biol 42: 819-32, Hellens R et al (2005) Plant Meth 1: 13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species. Transformation strategies may be designed to reduce, or eliminate, expression of a polynucleotide/polypeptide in a plant cell, tissue, organ or at a particular developmental stage which/when it is normally expressed. Such strategies are known as gene silencing strategies.

Genetic constructs for expression of genes in transgenic plants typically include promoters for driving the expression of one or more cloned polynucleotide, terminators and selectable marker sequences to detest presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference. Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

Gene Silencing

As discussed above, strategies designed to reduce, or eliminate, expression of a polynucleotide/polypeptide in a plant cell, tissue, organ, or at a particular developmental stage which/when it is normally expressed, are known as gene silencing strategies.

Gene silencing strategies may be focused on the gene itself or regulatory elements which effect expression of the encoded polypeptide. “Regulatory elements” is used here in the widest possible sense and includes other genes which interact with the gene of interest.

Genetic constructs designed to decrease or silence the expression of a polynucleotide/polypeptide of the invention may include an antisense copy of all or part a polynucleotide described herein. In such constructs the polynucleotide is placed in an antisense orientation with respect to the promoter and terminator. An “antisense” polynucleotide is obtained by inverting a polynucleotide or a segment of the polynucleotide so that the transcript produced will be complementary to the mRNA transcript of the gene, e.g.,

| | |
|---|---|
| 5′GATCTA 3′ (coding strand) | 3′CTAGAT 5′ |
| | (antisense strand) |
| 3′CUAGAU 5′ mRNA | 5′GAUCUCG 3′ antisense |
| | RNA |

Genetic constructs designed for gene silencing may also include an inverted repeat. An ‘inverted repeat’ is a sequence that is repeated where the second half of the repeat is in the complementary strand, e.g.,

5′-GATCTA . . . TAGATC-3′
3′-CTAGAT . . . ATCTAG-5′

The transcript formed may undergo complementary base pairing to form a hairpin structure. Usually a spacer of at least 3-5 bp between the repeated region is required to allow hairpin formation.

Such constructs are used in RNA interference (RNAi) approaches.

Another silencing approach involves the use of a small antisense RNA targeted to the transcript equivalent to an miRNA (Llave et al., 2002, Science 297, 2053). Use of such small antisense RNA corresponding to polynucleotide of the invention is expressly contemplated.

Transformation with an expression construct, as herein defined, may also result in gene silencing through a process known as sense suppression (e.g. Napoli et al., 1990, Plant Cell 2, 279; de Carvalho Niebel et al., 1995, Plant Cell, 7, 347). In some cases sense suppression may involve over-expression of the whole or a partial coding sequence but may also involve expression of non-coding region of the gene, such as an intron or a 5′ or 3′ untranslated region (UTR). Chimeric partial sense constructs can be used to coordinately silence multiple genes (Abbott et al., 2002, Plant Physiol. 128(3): 844-53; Jones et al., 1998, Planta 204: 499-505). The use of such sense suppression strategies to silence the target polynucleotides/genes is also contemplated.

The polynucleotide inserts in genetic constructs designed for gene silencing may correspond to coding sequence and/or non-coding sequence, such as promoter and/or intron and/or 5′ or 3′-UTR sequence, or the corresponding gene.

Preferably the insert sequence for use in a construct (e.g. an antisense, sense suppression or RNAi construct) for silencing of a target gene, comprises an insert sequence of at least 21 nucleotides in length corresponding to, or complementary, to the target gene.

Other gene silencing strategies include dominant negative approaches and the use of ribozyme constructs (McIntyre, 1996, Transgenic Res, 5, 257). Pre-transcriptional silencing may be brought about through mutation of the gene itself or its regulatory elements. Such mutations may include point mutations, frameshifts, insertions, deletions and substitutions.

Several further methods known in the art may be employed to alter, reduce or eliminate expression of a polynucleotide and/or polypeptide according to the invention. Such methods include but are not limited to Tilling (Till et al., 2003, Methods Mol Biol, 2%, 205), so called “Deletagene” technology (Li et al., 2001, Plant Journal 27(3), 235) and the use of artificial transcription factors such as synthetic zinc finger transcription factors. (e.g. Jouvenot et al., 2003, Gene Therapy 10, 513). Additionally antibodies or fragments thereof, targeted to a particular polypeptide may also be expressed in plants to modulate the activity of that polypeptide (Jobling et al., 2003, Nat. Biotechnol., 21(1), 35). Transposon tagging approaches may also be applied. Additionally peptides interacting with a polypeptide of the invention may be identified through technologies such as phase-display (Dyax Corporation). Such interacting peptides may be expressed in or applied to a plant to affect activity of a polypeptide of the invention. Use of each of the above approaches in alteration of expression of a nucleotide and/or polypeptide of the invention is specifically contemplated.

Methods for Modifying Endogenous DNA Sequences in Plant

Methods for modifying endogenous genomic DNA sequences in plants are known to those skilled in the art. Such methods may involve the use of sequence-specific nucleases that generate targeted double-stranded DNA breaks in genes of interest. Examples of such methods for use in plants include: zinc finger nucleases (Curtin et al., 2011. Plant Physiol. 156:466-473; Sander, et al., 2011. Nat. Methods 8:67-69.), transcription activator-like effector nucleases or “TALENs” (Cermak et al., 2011, Nucleic Acids Res. 39:e82; Mahfouz et al., 2011 Proc. Natl. Acad. Sci. USA 108:2623-2628; Li et al., 2012 Nat. Biotechnol. 30:390-392), and LAGLIDADG homing endonucleases, also termed “meganucleases” (Tzfira et al., 2012. Plant Biotechnol. J. 10:373-389).

In certain embodiments of the invention, one of these technologies (e.g. TALENs or a Zinc finger nuclease) can be used to modify one or more base pairs in a target gene to disable it, so it is no longer transcribaable and/or translatable.

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of biomedically important cell types and in organisms that have traditionally been challenging to manipulate genetically. A modified version of the CRISPR-Cas9 system has been developed to recruit heterologous domains that can regulate endogenous gene expression or label specific genomic loci in living cells (Nature Biotechnology 32, 347-355 (2014). The system is applicable to plants, and can be used to regulate expression of target genes. (Bortesi and Fischer, Biotechnology Advances Volume 33, Issue 1, January-February 2015, Pages 41-52).

Those skilled in the art will thus appreciate that there are numerous ways in which expression of target genes/polynucleotides/polypeptides can be reduced or eliminated. Any such method is included within the scope of the invention.

Transformation Protocols

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9: 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci.104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24(1):45-51); Prunus (Ramesh et al., 2006 Plant Cell Rep. 25(8):821-8; Song and Sink 2005 Plant Cell Rep. 2006; 25(2):117-23; Gonzalez Padilla et al., 2003 Plant Cell Rep.22(1):38-45); strawberry (Oosumi et al., 2006 Planta. 223(6):1219-30; Folta et al., 2006 Planta April 14; PMID: 16614818), rose (Li et al., 2003), Rubus (Graham et al., 1995 Methods Mol Biol. 1995;44:129-33), tomato (Dan et al., 2006, Plant Cell Reports V25:432-441), apple (Yao et al., 1995, Plant Cell Rep. 14, 407-412) and Actinidia eriantha (Wang et al., 2006, Plant Cell Rep. 25,5: 425-31). Transformation of other species is also contemplated by the invention. Suitable other methods and protocols are available in the scientific literature.

Plants

The term “plant” is intended to include a whole plant, any part of a plant, propagules and progeny of a plant.

The term ‘propagule’ means any part of a plant that may be used in reproduction or propagation, either sexual or asexual, including seeds and cuttings.

The plants of the invention may be grown and either self-ed or crossed with a different plant strain and the resulting off-spring from two or more generations also form an aspect of the present invention. Preferably the off-spring retain the construct, transgene or modification according to the invention.

General

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In certain embodiments the term “comprising” and related terms such as “comprise” and “comprises”, can be replaced with “consisting” and related terms, such as “consist” and “consists”.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

The present invention will be better understood with reference to the accompanying drawings in which:

FIG. 1 shows the Cluster of AGAMOUS like MADS box genes in Arabidopsis and Apple

FIG. 2 shows an alignment of MdAG (SEQ ID NO:1) with AtAG (SEQ ID NO:X)

FIG. 3 shows an alignment of MdAG (SEQ ID NO:1) with published MdMADS15 (SEQ ID NO:30).

FIG. 4 shows expression analysis of AG-like genes in untransformed (WT) apple and 2 independent ag RNAi transgenic lines showing ag phenotype (AS2905 and AS2921)

FIG. 5 shows the floral phenotype of suppression of AG in apples. ag(AS2921) mutants show whorls of petals and sepals.

FIG. 6 shows generation of apple through the treatment of ag (AS205) flowers with GA/IAA. These apples have reduced core tissue pushed towards the calex

FIG. 7 shows generation of apple through the treatment of ag (AS2921) flowers with GA/IAA/cytokinin. This apple has no apparent core tissue.

FIG. 8 shows a map of pTKO2S_262928, the MdAG sequences are shown as green arrows (KO seq)

FIG. 9 shows the conserved MADS domain and K domain of proteins MdAG (SEQ ID NO:1), MdPI (SEQ ID NO: 7), MdTM6 (SEQ ID NO: 13) and MdMADS13 (SEQ ID NO: 14).

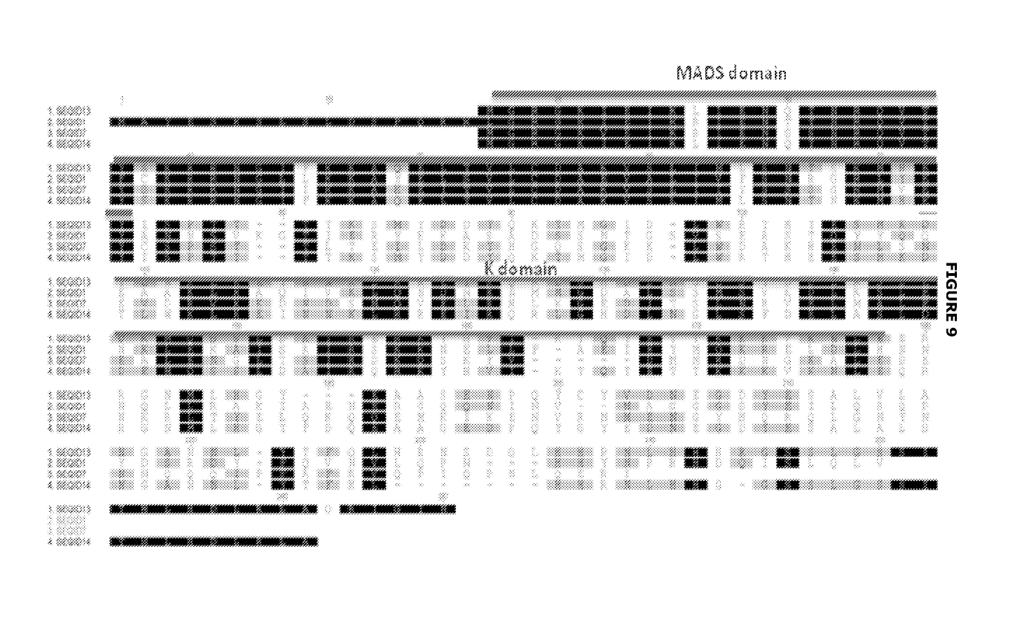

### EXAMPLES

The invention will now be illustrated with reference to the following non-limiting example.

It is not the intention to limit the scope of the invention to the abovementioned example only. As would be appreciated by a skilled person in the art, many variations are possible without departing from the scope of the invention.

### Example 1

### Production of Coreless Fruit by Reducing Expression of the Agamous (AG) Gene and Hormone Application

Gene Identification

The AG cluster in Arabidopsis consists of 4 genes which are AG, SEEDSTICK (STK) and SHATTERPROOF (SHP) 1 and 2. The ancient genome duplication in apples means that for each of these Arabidopsis genes there are two similar apple genes

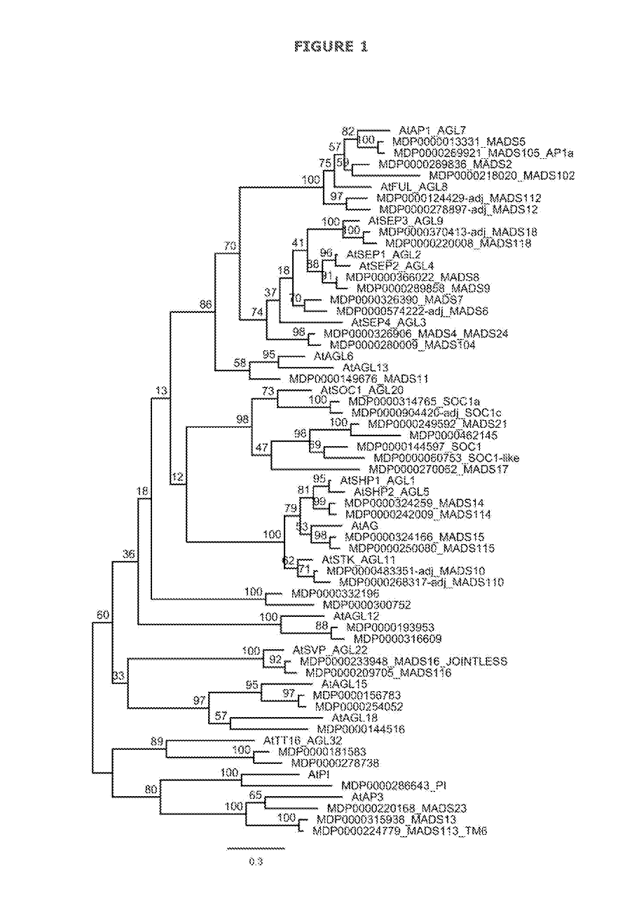

Using the apple genome (Velasco et al., 2010), apples MDP0000324166 and a homeologous gene MDP0000250080) are the most similar to Arabidopsis AG (atAG), see FIG. 1.

The first MDP0000324166, has been published as MADS15 (SEQ ID NO:30, van der Linden et al., 2002) The DNA sequence encoding MADS15 is shown in SEQ ID NO:31).

The applicants identified the equivalent gene for the apple cultivar Royal Gala, and designated this gene MdAG. The sequence of the MdAG protein and the polynucleotide encoding the protein are shown in SEQ ID NO: 1 and 4 respectively

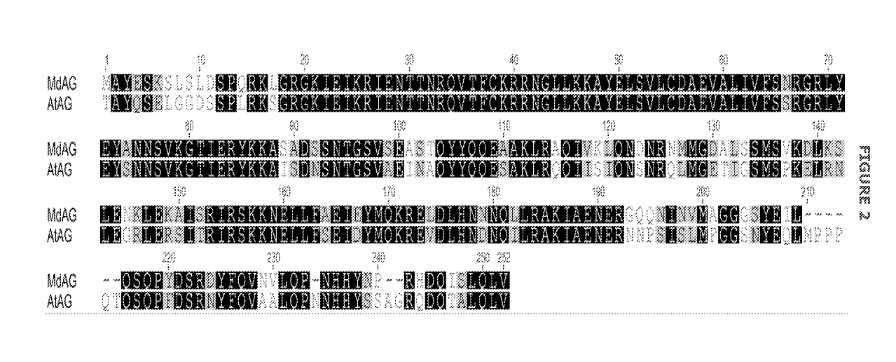

An alignment of MdAG and AtAG proteins is shown in FIG. 2.

Expression Analysis

Expression analysis of the gene as per mRNA seq of developing (balloon stage) flowers and open flowers show that MDP0000324166/MADS15/MdAG is higher expressed in apple flowers compared to MDP0000250080 MADS115.

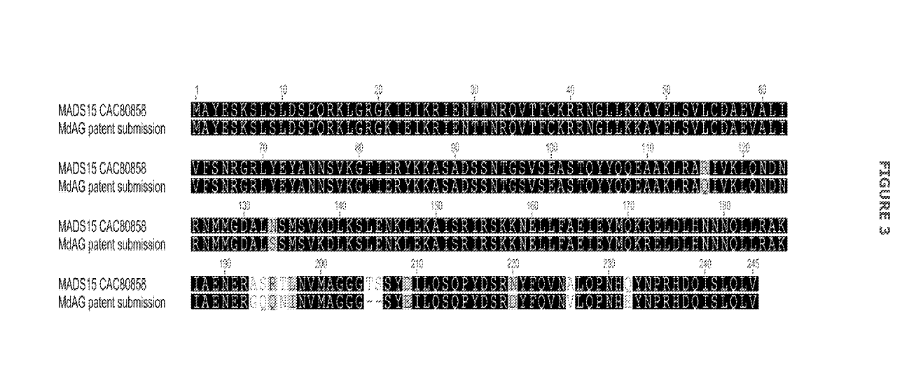

Suppression by RNAi of the MDP0000324166/MADS15/MdAG resulted in less transcript abundance (FIG. 3) and also down regulation of STK-like and SHP-like genes. (which maybe expected as these are both downstream of AG in Arabidopsis defining different carpel structures.) The next most similar genes outside this clade (SOC like) were unaffected (FIG. 3).

Creation of Plants Suppressed for MdAG

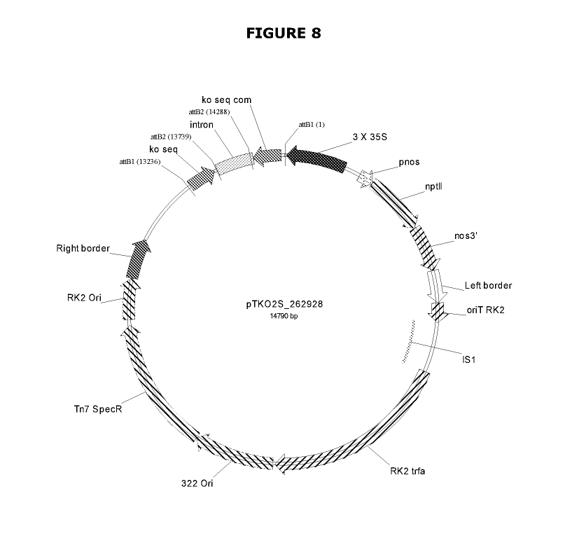

A hairpin construct, containing the first 403 bp (ATGGCCTATGAAAGCAAATCCTTGTCCTTGGACTCTCCCCAGAGAAAATTGGGTAGGGG AAAGATCGAGATTAAGCGGATCGAAAACACAACGAATCGTCAAGTGACCTTCTGCAAGA GGCGCAATGGGTTGCTCAAGAAGGCCTATGAACTCTCTGTGCTCTGTGATGCAGAGGTT GCTCTCATAGTCTTCTCTAACCGTGGCCGCCTCTATGAGTATGCCAACAATAGTGTTAAA GGAACAATTGAGAGGTACAAGAAGGCAAGTGCAGATTCTTCAAATACTGGATCAGTTTCT GAAGCTAGTACTCAGTACTACCAGCAAGAAGCTGCGAAATTGCGTGCGCAGATAGTGAA ATTGCAGAATGACAACCGGAATATGATGGGTGATGCATTGAGTAGTATGTCTGTCAAGG ACCTGAAGAGCCTGG—SEQ ID NO:25) of the MdAG gene of SEQ ID NO:4 was cloned into the a pDONOR (Invitrogen) and inserted into the gateway compatible pTKO2 vector (Snowden et al., 2005) in an inverted repeat (green KO seq—FIG. 8). These were transformed into ‘Royal Gala’ apples as described by (Yao et al., 1995).

Construction of hairpin knockout vector pTKO2S_262928 (EST 262928)

The hairpin knockout vector pTKO2S_262928 (EST 262928) was constructed with pTKO2 (Snowden et al 2005) using Gateway Technology (Invitrogen).

PCR was carried out on pBluescript (SK-) EST_262928 with the primers 262928_F (Gateway attB1—atggcctatgaaagcaaatcc—SEQ ID NO:26) and 262928_R (Gateway attB2—CCAGGCTCTTCAGGTCCTTG—SEQ ID NO:27) to give a PCR product of 430 bp.

Amplfication was carried out on 10 ng of template DNA with 0.5 mM of each primer, 0.8 mM dNTPs, 1× Taq DNA polymerase buffer, 0.5 U Expand High Fidelity Taq DNA polymerase (Boehringer Mannheim) in a Techne Progene cycler: 94° C. (3 min), followed by 30 cycles of 94° C. (30 s), 60° C. (45 s), 68° C. (1 min).

The Gateway BP reaction with PCR product and pDONR was carried out as recommended by the manufacturer (Invitrogen). Plasmid DNA of resulting transformants was isolated using Wizard Plus Miniprep DNA Purification System (Promega) and the correct constructs were verified by restriction enzyme analysis for the pENTRY_262928 (430 bp insert).

Gateway LR reactions with the resulting pENTRY_262928 vector and destination vector pTKO2 was carried out as recommended by the manufacturer. (Invitrogen).

The final construct was verified by restriction enzyme analysis.

A map of pTKO2S_262928 is shown in FIG. 8.

Phenotype of the RNAi Suppressed Lines

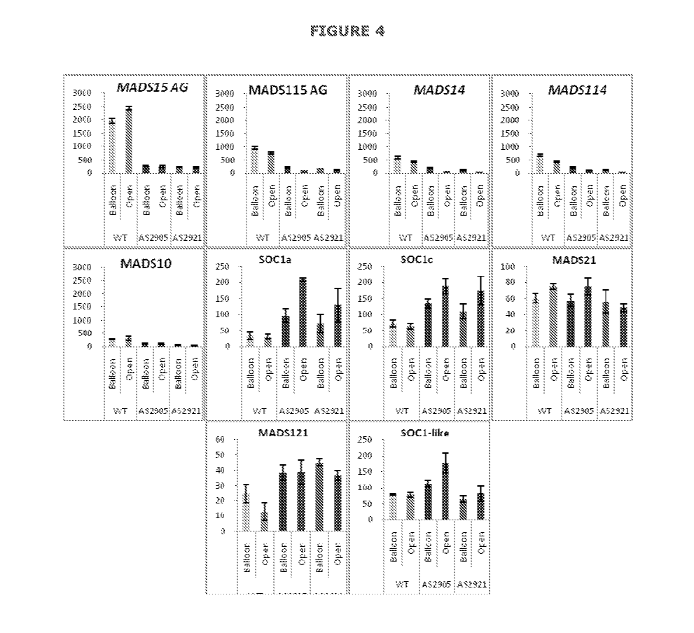

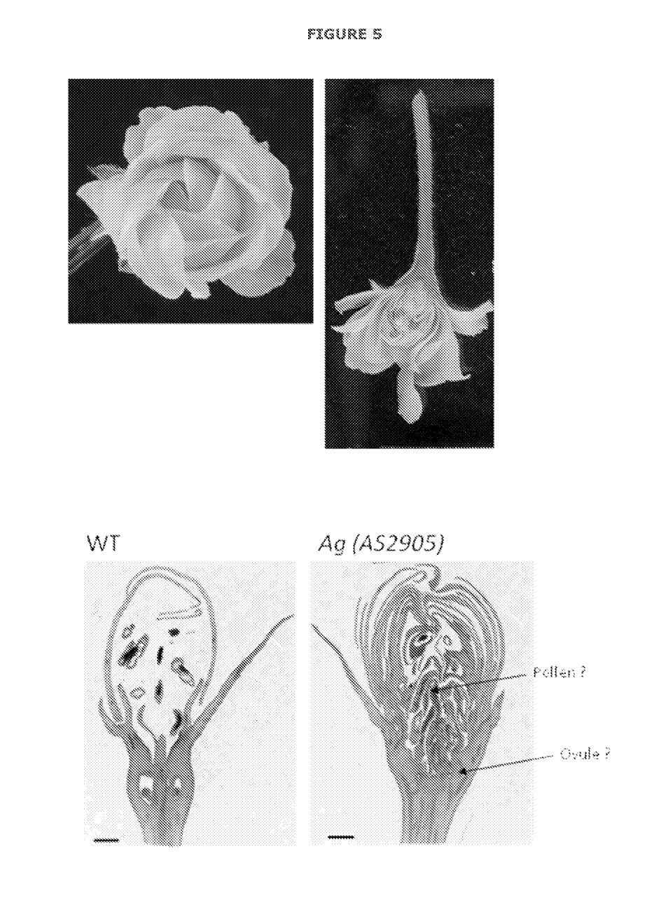

Apples with suppressed AG have floral conversion to whorls of sepals and petals these can be seen in FIG. 4. This is consistent with the literature when you knock out AG in other species such as Arabidopsis (Yanofsky et al., 1990). Microscopy of one of the suppressed lines (AS2905) revealed that there are apparent remnant ovules and possible pollen like formations in the more acropetal whorls of organs (FIG. 5)

Induction of Parthenocarpy

Pathenocarpy (production of fruit with no pollination) can be induced with hormone treatment or genetically with the modulation of certain genes detailed in (Sotelo-Silveira et al., 2014).

In apples extensive work was done to induce parthenocarpy, only the triple combination of GA3, SD8339, and 2-NAA, rather than single or paired application, resulted in parthenocarpy in Cox's Orange Pippin (Kotob and Schwabe, 1971) and GA4+7 alone induced parthenocarpy in frost-damaged Bramley's Seedling and cytokinin SD8339 had no additional benefits; GA3 was not effective. This said, Bramley's Seedling is triploid and partially self-fertile so may be an unusual case (Modlibowska, 1972).

To induce parthenocarpy in the ag apples, treatments with different concentrations and combinations of Gibberellins (GA), Auxin (IAA) and Cytokinins (BAP) were applied to the flowers. Hormone concentrations: 300 ppm GA4 & 1 ppm IAA, and 300 ppm GA4, 100 ppm 6-BA, & 1 ppm IAA.

All treatments started at a stage around −7DAFB. All flowers treated −7, −4 and +1 DAFB, three treatments in total for most flowers, 4 treatments for a few.

| | | | | final fruit | |
|---|---|---|---|---|---|
| Genotype | treatment | infor. | flowers | numbers | |
| wild-type | GA/IAA | 22 | 110 | 5 | 4.5% |
| | GA/IAA/BA | 23 | 115 | 7 | 6.1% |
| AS2905 | GA/IAA | 22 | 110 | 1 | 0.9% |
| AS2921 | GA/IAA/BA | 8 | 40 | 1 | 2.5% |

These apples were allowed to grow to maturity, then they were harvested and assessed for presence of core. Apples from transgenic lines containing partial ovules were able to be induced with GA and IAA alone. Transgenic lines with more severe phenotype (no ovule tissue) needed cytokinins (FIG. 5).

Properties of Reduced Core and Coreless Apples

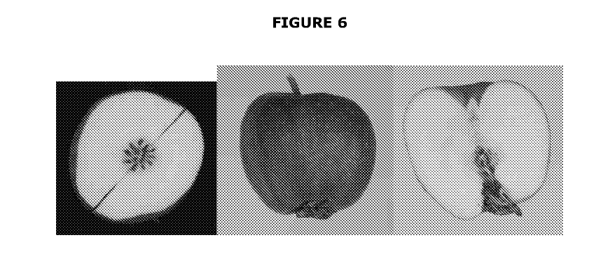

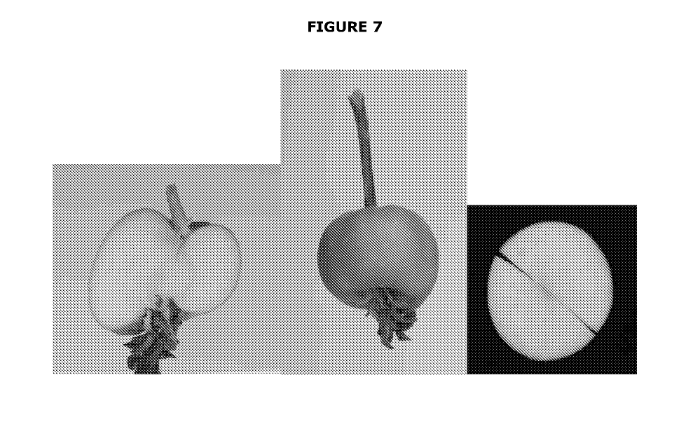

Reduced core and coreless apples are shown in FIGS. 6 and 7 respectively. With reduced cored apples (FIG. 6) having less locule tissue and an increase in relative amounts of flesh tissue compared to untransformed controls. With the complete absence of ovule (Core) tissue (FIG. 7), no locules or seed bearing tissue is present and the flesh tissue is distributed throughout the apple.

### Example 2

### Production of Coreless Fruit by Reducing Expression of AG and AP3-Like Genes

It will be understood by those skilled in the art that apple plants that do no express AP3-like genes are parthenocarpic (Yao et al. 2001). Therefore in accordance with the present invention, suppression of both AG and AP3-like genes (to induce parthenocarpy) results in plants than produce coreless fruit.

Hairpin Construct for Suppressing AP3-Like Genes

To suppress the two apple AP3-like genes, MdMADS13 and MdTm6, a hairpin construct containing the first 414 bp (ATATATCAAGTAAAACAAGATCAGAAAATTGCTAGGAAAAGGTAAGAAATTTGAGAGAG AGAGAGAAATTATGGGTCGTGGGAAGATTGAAATCAAGCTGATCGAAAACCAGACCAAC AGGCAGGTGACCTACTCCAAGAGAAGAAATGGGATCTTCAAGAAGGCTCAGGAGCTCAC CGTTCTCTGTGATGCCAAGGTCTCCCTCATTATGCTCTCCAACACTAATAAAATGCACGA GTATATCAGCCCTACCACTACGACCAAGAGTATGTATGATGACTATCAGAAAACTATGGG GATCGATCTGTGGAGGACACACGAGGAGTCGATGAAAGACACCTTGTGGAAGTTGAAAG AGATCAACAATAAGCTGAGGAGAGAGATCAGGCAGAGGTTGGGCCATGATCTAAATGG—SEQ ID NO:28) of MdMADS13 (SEQ ID NO:20) can be cloned into the a pDONOR (Invitrogen) and inserted into the gateway compatible pTKO2 vector (Snowden et al., 2005) as an inverted repeat. This construct will suppress both MdTM6 and MdMADS13 because the DNA sequences in this region are highly conserved between the two genes.

Transformation

To suppress both AG and the AP3-like genes, this construct and the MdAG suppressing construct (described in Example 1) can both be transformed into ‘Royal Gala’ apples as described in Example 1 and Yao et al., 1995.

Transgenic plants containing both gene constructs can be identified using PCR analysis and grown in a glasshouse for fruit production and phenotype analysis.

This will result in an apple plant with reduced, or eliminated, expression of both MdAG and the AP3-like genes, which will produce coreless fruit.

### Example 3

### Production of Coreless Fruit by Reducing Expression of AG and PI Genes

It will be understood by those skilled in the art that apple plants that do no express PI genes are parthenocarpic (Yao et al. 2001). Therefore according to the invention, suppression of both AG and PI genes results in plants than produce coreless fruit.

Hairpin Construct for Suppressing PI Genes

To suppress the two apple MdPI, a hairpin construct containing the first 414 bp (ATGGGACGTGGGAAGGTTGAGATCAAGAGGATTGAGAACTCAAGTAACAGGCAGGTGA CCTACTCCAAGAGGAGGAATGGGATTATCAAGAAGGCAAAGGAGATCACTGTTCTATGT GATGCTAAAGTATCTCTTATCATTTATTCTAGCTCTGGGAAGATGGTTGAATACTGCAGC CCTTCAACTACGCTGACAGAAATCTTGGACAAATACCATGGACAATCTGGGAAGAAGTTG TGGGATGCTAAGCATGAGAACCTCAGCAATGAAGTGGATAGAGTCAAGAAAGACAATGA CAGCATGCAAGTAGAGCTCAGGCATCTGAAGGGAGAGGATATCACATCATTGAACCATG TAGAGCTGATGGCCTTAGAGGAAGCACTTGAAAATGGCCTTACAAGTATCCGGGACAAG—SEQ ID NO:29) of MdPI (SEQ ID NO:20) can be cloned into pDONOR (Invitrogen) and inserted into the gateway compatible pTKO2 vector (Snowden et al., 2005) as an inverted repeat.

Transformation

To suppress both MdAG and the MDPI genes, this construct and the MdAG suppressing construct (described in Example 1) can both be transformed into ‘Royal Gala’ apples as described in Example 1 and Yao et al., 1995.

Transgenic plants containing both gene constructs can be identified using PCR analysis and grown in a glasshouse for fruit production and phenotype analysis.

This will result in an apple plant with reduced, or eliminated, expression of both MdAG and the MdPI genes, which will produce coreless fruit.

### Example 4

### Production of Coreless Fruit by Reducing Expression of AG in a Pistilata (PI) Mutant

It will be understood by those skilled in the art that apple plants that do no express MdPI genes are parthenocarpic (Yao et al. 2001). Therefore in accordance with the present invention, suppression of AG in a plant that does not express a PI gene results in plants than produce coreless fruit.

The hairpin construct designed to suppress MdAG (described in Example 1, and shown in FIG. 8) can be transferred into the ‘Rae Ime’ apple mutant (for example) that does not express the apple MdPI gene (Yao et al. 2001) using the method as described in Example 1.

This will result in an apple plant with reduced, or eliminated, expression of both MdAG and MdPI which will produce coreless fruit.

### Example 5

### Production Plants Producing Coreless Fruit by Non-Transgenic Means

In accordance with the invention apple plants with suppressed or eliminated expression if AG and PI, or AG and AP3-like genes will produce coreless fruit.

Apple plants with reduced, or eliminated, expression of both AG and PI can be produced by combining natural apple mutants using sexual crossing. First, natural mutants of apple AG gene are identified. The AG suppressed apples have increased whorls of petals and can therefore be selected amongst existing cultivars.

The applicants have identified, within their germplasm collections, natural mutants of AG with apple varieties, such as Malus ioensis ‘Plena’, which show a similar phenotype to the AG suppression transgenic plants, described in Example 1.

Plants with such a phenotype can optionally be selected for whole genome sequencing to identify mutations in the AG genes, and for q-RT-PCR analysis to confirm the reduced or eliminated expression of the AG gene. Alternatively plants can be screened for reduced expression of the AG gene first.

The AG mutant plant can be crossed with parthenocarpic plants, such as the PI mutants described herein, by methods well known to those skilled in the art.

For example the AG and PI mutants can for example be combined with high fruit quality by rapid introgression breeding using a fast flowering ‘Royal Gala’ apple line. A ‘Royal Gala’ apple transgenic line has been established by over-expression of a flowering promotion gene. This line flowered a few weeks after transplanted into greenhouse from tissue culture. Seedlings of this line would be expected to flower within one year, i.e. one year per generation compared to 6-8 years per generation for normal apple plants.

Resulting plants with reduced, or eliminated, expression of both MdAG and MdPI which will produce coreless fruit.

If the varieties containing AG and PI mutaions are poor in fruit quality, multiple of back-crosses to premium apple cultivars can be performed, by methods well known to those skilled in the art, in order to maintain the high fruit quality of the future coreless apple cultivars.

### REFERENCES

Kotob M, Schwabe W. 1971. Induction of parthenocarpic fruit in Cox's Orange Pippin apples. J Hort Sci.

Modlibowska I. 1972. effect of gibberellins and cytokinins on fruit development of Bramley's Seedling apple. J Hort Sci.

Snowden K C, Simkin A J, Janssen B J, Templeton K R, Loucas H M, Simons J L, Karunairetnam S, Gleave A P, Clark D G, Klee H J. 2005. The Decreased apical dominance1/Petunia hybrida CAROTENOID CLEAVAGE DIOXYGENASE8 Gene Affects Branch Production and Plays a Role in Leaf Senescence, Root Growth, and Flower Development. The Plant Cell Online 17, 746-759.

Sotelo-Silveira M, Marsch-Martinez N, de Folter S. 2014. Unraveling the signal scenario of fruit set. Planta 239, 1147-1158.

van der Linden C G, Vosman B, Smulders M J M. 2002. Cloning and characterization of four apple MADS box genes isolated from vegetative tissue. Journal of Experimental Botany 53, 1025-1036.

Velasco R, Zharkikh A, Affourtit J, Dhingra A, Cestaro A, Kalyanaraman A, Fontana P, Bhatnagar S K, Troggio M, Pruss D. 2010. The genome of the domesticated apple (Malus [times] domestica Borkh.). Nature genetics 42, 833-839.

Yanofsky M F, Ma H, Bowman J L, Drews G N, Feldmann K A, Meyerowitz E M. 1990. The protein encoded by the Arabidopsis homeotic gene agamous resembles transcription factors. Nature 346, 35-39.

Yao J-L, Cohen D, Atkinson R, Richardson K, Morris B. 1995. Regeneration of transgenic plants from the commercial apple cultivar Royal Gala. Plant Cell Reports 14, 407-412.

Yao, J.-L., Dong, Y.-H. & Morris, B. A. Parthenocarpic apple fruit production conferred by transposon insertion mutations in a MADS-box transcription factor. Proceedings of the National Academy of Sciences 98, 1306-1311 (2001).

1-42. (canceled)

43. A method for producing a coreless fruit, or a plant that produces at least one coreless fruit, the method comprising reducing, or eliminating, expression of at least one AGAMOUS (AG) protein in a plant from a species that produces accessory fruit.

44. The method of claim 43 that includes the additional step of inducing parthenocarpy in the plant.

45. The method of claim 43 wherein the plant in which expression of at least one AGAMOUS (AG) protein is reduced or eliminated, is a parthenocarpic plant.

46. The method of claim 44 in which parthenocarpy is induced by application of plant hormones to flowers of the plant.

47. The method of claim 44 in which parthenocarpy is induced by manipulating expression of genes controlling fruit set.

48. The method of claim 47 in which parthenocarpy is induced by reducing, or eliminating expression, of at least one PISTILLATA (PI) gene or protein.

49. The method of claim 47 in which parthenocarpy is induced by reducing, or eliminating expression, of at least one APETALA3 (AP3) gene or protein.

50. The method of claim 45 in which the parthenocarpic plant is a mutant plant with reduced, or eliminated expression, of at least one PISTILLATA (PI) gene or protein.

51. The method of claim 45 in which the parthenocarpic plant is a mutant plant with reduced, or eliminated, expression of at least one APETALA3 (AP3) gene or protein.

52. A method for producing a coreless fruit, or a plant that produces at least one coreless fruit, the method comprising reducing, or eliminating, in a plant from a species that produces accessory fruit, expression of:
a) at least one AGAMOUS (AG) protein, and
b) at least one of:
i) at least one PISTILLATA (PI) protein, and
ii) at least one APETALA3 (AP3) protein

53. the method of claim 52 in which reducing, or eliminating, expression of the PISTILLATA (PI) protein or APETALA3 (AP3) protein induces parthenocarpy.

54. A method of detecting, in a plant from a species that produces accessory fruit, at least one of:
a) reduced, or eliminated, expression of at least one AGAMOUS (AG) protein,
b) reduced, or eliminated, expression of at least one polynucleotide encoding an AGAMOUS (AG) protein,
c) presence of a marker associated with reduced expression of at least one AGAMOUS (AG) protein, and
d) presence of a marker associated with reduced expression of at least one polynucleotide encoding an AGAMOUS (AG) protein.

55. The method of claim 54 wherein detecting any of a) to d) indicates that the plant will produce, or be useful for producing, at least one coreless fruit.

56. The method of claim 54, wherein plant identified is a mutant plant with reduced or eliminated expression of an AGAMOUS (AG) gene or protein.

57. The method of claim 54, further comprising detecting in the plant, at least one of:
e) reduced, or eliminated, expression of at least one PISTILLATA (PI) or APETALA3 (AP3) protein,
f) reduced, or eliminated, expression of at least one polynucleotide encoding a PISTILLATA (PI) or APETALA3 (AP3) protein,
g) presence of a marker associated with reduced expression of at least one PISTILLATA (PI) or APETALA3 (AP3) protein, and
h) presence of a marker associated with reduced expression of at least one polynucleotide encoding a PISTILATA (PI) or APETALA3 (AP3) protein.

58. The method of claim 57 wherein detecting any of e) to h) indicates that the plant will produce, or be useful for producing, at least one coreless fruit.

59. The method of claim 57, wherein plant identified is a mutant plant with reduced or eliminated expression of a PISTILLATA (PI) or APETALA3 (AP3) gene or protein.

60. A method for producing a plant that produces at least one coreless fruit, the method comprising crossing at least one of:
a) a plant with reduced, or eliminated, expression of at least one AGAMOUS (AG) protein, and
b) a mutant plant with reduced, or eliminated, expression of at least on one of AGAMOUS (AG) protein,
with another plant, wherein the off-spring produced by the crossing is a plant that produces at least one coreless fruit, and wherein the plant in a), the plant in b) and the another plant are from a species that produces accessory fruit.

61. The method of claim 60 in which at least one of the plant of a), the plant of b, and the another plant, is at least one of:
i) a parthenogenic plant,
ii) a plant with reduced or eliminated expression of at least one PISTILLATA (PI) protein,
iii) a plant with reduced or eliminated expression of at least one APETALA3 (AP3) protein.

62. A method for producing a coreless fruit, the method comprising cultivating a plant in which at least one of a) to d) is detected in the method of claim 54.

63. The method of claim 62 which includes the additional step of inducing parthenocarpy in the plant.

64. The method of claim 62 in which the plant produces coreless fruit as a result of having reduced, or eliminated expression, of at least one AGAMOUS (AG) protein, and having reduced, or eliminated expression, of one of an PISTILLATA (PI) protein and an APETALA3 (AP3) protein.

65. A method of producing a coreless fruit the method comprising cultivating a plant, from a species that produces accessory fruit, with reduced, or eliminated, expression of at least one AGAMOUS (AG) protein.

66. The method of claim 65 wherein the plant also has reduced, or eliminated, expression of at least one of:
i) at least one PISTILLATA (PI) protein, and
ii) at least one APETALA3 (AP3) protein.

67. A coreless fruit produced by a method of claim 65.

68. A coreless fruit, or plant that produces at least one coreless fruit, with reduced or eliminated expression of at least one AGAMOUS (AG) protein, wherein the coreless fruit or plant is from a species that produces accessory fruit.

69. The coreless fruit, or plant of claim 68 that also has reduced or eliminated expression of at least one of:
i) at least one PISTILLATA (PI) protein, and
ii) at least one APETALA3 (AP3) protein.

70. A coreless fruit or a plant of claim 68, wherein the coreless fruit or plant comprises a construct for reducing, or eliminating, the expression, in a plant, of at least one of:
a) an AGAMOUS (AG) protein,
b) a PISTILLATA (PI) protein, and
c) an APETALA3 (AP3) protein.

71. The coreless fruit or plant of claim 70 wherein the construct comprises part of a gene or polynucleotide that encodes the protein.

72. The coreless fruit or plant of claim 70 wherein the construct is designed to reduce, or eliminate, expression of at least one of:
a) an AGAMOUS (AG) protein and a PISTILLATA (PI) protein, and
b) an AGAMOUS (AG) protein and an APETALA3 (AP3) protein.

73. Use of a plant part, progeny, or propagule of a plant of claim 66, that has reduced, or eliminated expression of at least on AGAMOUS (AG) protein to produce a plant that produces at least one coreless fruit.

74. The use of claim 73 in which plant part, progeny, or propagule also has reduced, or eliminated expression of at least one of:
a) a PISTILLATA (PI) protein, and
b) an APETALA3 (AP3) protein.


SEQUENCE LISTING (33 sequences)

SEQ ID NO: 1 - protein, 243 aa, Malus domestica
MAYESKSLSLDSPQRKLGRGKIEIKRIENTTNRQVTFCKRRNGLLKKAYELSVLCDAEVALIVFSNRGRLYEYANNSVKGTIERYKKASADSSNTGSVSEASTQYYQQEAAKLRAQIVKLQNDNRNMMGDALSSMSVKDLKSLENKLEKAISRIRSKKNELLFAEIEYMQKRELDLHNNNQLLRAKIAENERGQQNINVMAGGGSYEILQSQPYDSRDYFQVNVLQPNHHYNPRHDQISLQLV

SEQ ID NO: 2 - protein, 243 aa, Pyrus bretschneideri
MAYESKSLSMDSPQRKLGRGKIEIKRIENTTNRQVTFCKRRNGLLKKAYELSVLCDAEVALVVFSNRGRLYEYANNSVKGTIERYKKACADSSNTGSVSEASTQYYQQEAAKLRAQIVKLQNDNRNMMGDALSSMPVKDLKSLENKLEKGISRIRSKKNELLFAEIEYMQKRELDLHNNNQLLRAKIAENERGQQNINVMAGGGSYEILQSQPYDSRDYFQVNVLQPNNHYNPRHDQISLQLV

SEQ ID NO: 3 - protein, 243 aa, Pyrus communis
MAYESKSLSMDSPQRKLGRGKIEIKRIENTTNRQVTFCKRRNGLLKKAYELSVLCDAEVALIVFSNRGRLYEYANNSVKGTIERYKKACADSSNTGSVSEASTQYYQQEAAKLRAQIVKLQNDNRNMMGDALSSMSVKDLKSLENKLEKGISRIRSKKNELLFAEIEYMQKRELDLHNNNQLLRAKIAENERGQQNINVMAGGGSYEILQSQPYDSRDYFQVNVLQPNNHYNPRHDQISLQLV

SEQ ID NO: 4 - DNA, 923 bp, Malus domestica
atggcctatgaaagcaaatccttgtccttggactctccccagagaaaattgggtaggggaaagatcgagattaagcggatcgaaaacacaacgaatcgtcaagtgaccttctgcaagaggcgcaatgggttgctcaagaaggcctatgaactctctgtgctctgtgatgcagaggttgctctcatagtcttctctaaccgtggccgcctctatgagtatgccaacaatagtgttaaaggaacaattgagaggtacaagaaggcaagtgcagattcttcaaatactggatcagtttctgaagctagtactcagtactaccagcaagaagctgcgaaattgcgtgcgcagatagtgaaattgcagaatgacaaccggaatatgatgggtgatgcattgagtagtatgtctgtcaaggacctgaagagcctggagaataaactggagaaagcaattagcagaatccgatccaaaaagaatgagctcttgtttgccgaaattgagtacatgcagaaaagggaactggacttgcacaacaataaccagctcctacgagcaaagatagctgagaatgagaggggccagcagaacataaatgtgatggctggaggaggaagttatgagatcctgcagtctcagccatacgactctcgggactatttccaagtgaacgtgttacaacccaatcatcactacaatccccgccacgatcagatttcccttcaattagtatgaatgatcaagattgctttgggaatgaagaccgttggtaccaagtacgtaggcatacgtacgtattttagtatatgaaagcggatttctcgtctgtatttttatatttatgcctgcagcaatgtgtactactaagatttctgtgagaaattaccctaacaagttcctccaacttacatgtgaaaaaaaaaaaa

SEQ ID NO: 5 - DNA, 729 bp, Pyrus bretschneideri
atggcctatgaaagcaaatccttgtccatggactctccccagagaaaattgggtaggggaaagatcgagattaagcggatcgaaaacacgacaaatcgtcaagtgaccttctgcaagaggcgcaatgggttgctcaagaaggcctatgaactctctgtgctctgtgatgccgaggttgctctcgtagtcttctctaaccgtggccgcctctatgagtatgccaacaatagtgttaaaggaacaattgagaggtacaagaaggcatgtgcagattcttcaaatactggatcagtttctgaagctagcactcagtactaccagcaagaagctgcgaaattgcgtgcgcagatagtgaaattgcagaatgacaaccggaatatgatgggtgatgcattgagtagtatgcctgtcaaggacctgaagagcctggagaataaactggagaaaggaattagcagaatccgatccaaaaagaatgagctcttgtttgccgaaattgagtacatgcagaaaagggaactggacttgcacaacaataaccagctcctacgagcaaagatagctgagaatgagaggggccagcagaacataaatgtaatggctggaggaggaagctatgagatcctgcagtctcagccatacgactctcgggactatttccaagtgaacgtgttacaacccaataatcactacaatccccgccacgatcagatttcccttcaattagta

SEQ ID NO: 6 - DNA, 729 bp, Pyrus communis
atggcctatgaaagcaaatccttgtccatggactctccccagagaaaattgggtaggggaaagatcgagattaagcggatcgaaaacacgacaaatcgtcaagtgaccttctgcaagaggcgcaatgggttgcttaagaaggcctatgaactctctgtgctctgtgatgctgaggttgctctcatagtcttctctaaccgtggccgcctctatgagtatgccaacaatagtgttaaaggaacaattgagaggtacaagaaggcatgtgcagattcttcaaatactggatcagtttctgaagctagtactcagtactaccagcaagaagctgcgaaattgcgtgcgcagatagtgaaattgcagaatgacaaccggaatatgatgggtgatgcattgagtagtatgtctgtcaaggacctgaagagcctggagaataaactggagaaaggaattagcagaatccgatccaaaaagaatgagctcttgtttgccgaaattgagtacatgcagaaaagggaactggacttgcacaacaataaccagctcctacgagcaaagatagctgagaatgagaggggccagcagaacataaatgtaatggctggaggaggaagctatgagatcctgcagtctcagccatacgactctcgggactatttccaagtgaacgtgttacaacccaataatcactacaatccccgccacgatcagatttccctacaattagta

SEQ ID NO: 7 - protein, 215 aa, Malus domestica
MGRGKVEIKRIENSSNRQVTYSKRRNGIIKKAKEITVLCDAKVSLIIYSSSGKMVEYCSPSTTLTEILDKYHGQSGKKLWDAKHENLSNEVDRVKKDNDSMQVELRHLKGEDITSLNHVELMALEEALENGLTSIRDKQSKFVDMMRDNGKALEDENKRLTYELQKQQEMKIKENVRNMENGYHQRQLGNYNNNQQQIPFAFRVQPIQPNLQERI

SEQ ID NO: 8 - protein, 217 aa, Pyrus communis
MGRGKIEIKRIENSSNRQVTYSKRRNGIIKKAKEITVLCDAKVSLIIYSSSGKMVEYCSPSTTLTEILDKYHGQSGKKLWDAKHENLSNEVDRVKKDNDSMQVELRHLKGEDITSLNHLELMALEEALENGLTSIRDKQASFFHYSYFKQSGKALEDENKRLTYELQKQQEMKIDENVRNMENGYHQRQLGNYNNTQQQIPFAFRVQPIQPNLQERI

SEQ ID NO: 9 - protein, 215 aa, Pyrus bretschneideri
MGRGKIEIKRIENSSNRQVTYSKRRNGIIKKAKEITVLCDAKVSLIIYSSSGKMVEYCSPSTTLTEILDKYHGQSGKKLWDAKHENLSNEVDRVKKDNDSMQVELRHLKGEDITSLNHVELMALEEALENGLTSIRDKQSKFVDMMRDNGKALEDENKRLTYELQKQQEMKIGENVRNMENGYHQRQLGNYNNTQQQIPFAFRVQPIQPNLQERI

SEQ ID NO: 10 - DNA, 842 bp, Malus domestica
atgggacgtgggaaggttgagatcaagaggattgagaactcaagtaacaggcaggtgacctactccaagaggaggaatgggattatcaagaaggcaaaggagatcactgttctatgtgatgctaaagtatctcttatcatttattctagctctgggaagatggttgaatactgcagcccttcaactacgctgacagaaatcttggacaaataccatggacaatctgggaagaagttgtgggatgctaagcatgagaacctcagcaatgaagtggatagagtcaagaaagacaatgacagcatgcaagtagagctcaggcatctgaagggagaggatatcacatcattgaaccatgtagagctgatggccttagaggaagcacttgaaaatggccttacaagtatccgggacaagcagtccaagttcgtcgacatgatgagagacaatggaaaggcactggaagatgagaataagcgcctcacttatgagctgcaaaaacaacaggagatgaaaataaaagagaatgtgagaaacatggaaaatgggtatcatcagaggcagctggggaactacaacaacaaccagcagcagataccttttgccttccgcgtgcagcctattcagccaaatctccaggagagaatctaattagatatatcttgcatttgcatgctctttctaactagttatattatctctccacctctctctctcttttcatctgtcaaggagttcttaagtttatgtcagatttccaatggtttgtaatggaattagcttcgttatgaggctttgttgtgaaccttgtaataattaaggcgtgcatgaactcggtttgtggg

SEQ ID NO: 11 - DNA, 654 bp, Pyrus communis
atggggaggggtaagattgagatcaagaggattgagaactcaagtaacaggcaggtgacctactccaagaggaggaatgggattatcaagaaggcaaaggagatcactgttttatgtgatgctaaagtatctcttatcatttattctagctctgggaagatggttgaatactgcagcccttcaactacgctgacagaaatcttggataaataccatggccaatctgggaagaagttgtgggatgctaagcatgagaacctcagcaatgaagtggatagagtcaagaaagacaatgacagcatgcaagtagagctcaggcatctgaagggagaggatatcacatcattgaaccatttagagctgatggccttagaggaagcacttgaaaatggccttacaagtatccgggacaagcaggcaagtttcttccattattcatacttcaaacagagtggaaaggcactggaagatgagaataagcgcctcacttatgagctgcaaaaacaacaggagatgaaaatagatgagaatgtgagaaacatggaaaatgggtatcatcagaggcagctggggaactacaacaacacccagcagcagataccttttgccttccgcgtgcagcctattcagccaaatctccaggagagaatctaa

SEQ ID NO: 12 - DNA, 648 bp, Pyrus bretschneideri
atggggaggggtaagattgagatcaagaggattgagaactcaagtaacaggcaggtgacctactccaagaggaggaatgggattatcaagaaggcaaaggagatcactgttttatgtgatgctaaagtatctcttatcatttattctagctctgggaagatggttgaatactgcagcccttcaactacgctgacagaaatcttggataaataccatggccaatctgggaagaagttgtgggatgctaagcatgagaacctcagcaatgaagtggatagagtcaagaaagacaatgacagcatgcaagtagagctcaggcatctgaagggagaggatatcacatcattgaaccatgtagagctgatggccttagaggaagcacttgaaaatggccttacaagtatccgggacaagcagtccaagttcgtcgacatgatgagagacaatggaaaggcactggaagatgagaataagcgcctcacttatgagctgcaaaaacaacaggagatgaaaataggtgagaatgtgagaaacatggaaaatgggtatcatcagaggcagctgggaaactacaacaacacccagcagcagatacctttcgccttccgcgtgcagcctattcagccaaatctccaggagagaatctaa

SEQ ID NO: 13 - protein, 242 aa, Malus domestica
MGRGKIEIKLIENQTNRQVTYSKRRNGIFKKAQELTVLCDAKVSLIMLSNTSKMHEYISPTTTTKSMYDDYQKTMGIDLWRTHYESMKDTLWKLKEINNKLRREIRQRLGHDLNGLSYDDLRSLEDKMQSSLDAIRERKYHVIKTQTETTKKKVKNLEERRGNMLHGYEAASENPQYCYVDNEGDYESALVLANGANNLYTFQLHRNSDQLHHPNLHHHRGSSLGSSITHLHDLRLARIGIN

SEQ ID NO: 14 - protein, 232 aa, Malus domestica
MGRGKIEIKLIENQTNRQVTYSKRRNGIFKKAQELTVLCDAKVSLIMLSNTNKMHEYISPTTTTKSMYDDYQKTMGIDLWRTHEESMKDTLWKLKEINNKLRREIRQRLGHDLNGLSFDELASLDDEMQSSLDAIRQRKYHVIKTQTETTKKKVKNLEQRRGNMLHGYFDQEAAGEDPQYGYEDNEGDYESALALSNGANNLYTFHLHHRNLHHGGSSLGSSITHLHDLRLA

SEQ ID NO: 15 - protein, 240 aa, Pyrus bretschneideri
MGRGKIEIKLIENQTNRQVTYSKRRNGIFKKAQELTVLCDAKVSLIMLSNTSKMHEYISPTTTTKRMYDDYQKTMGVDLWRTHYESMKDTLWKLKEINNKLRREIRQRLGHDLNGLSYDDLRSLEDKMQSSLDAIRERKYHVIKTQTETTKKKVKNLEERRGNMLHGYFDQEAASENPQYCYVDNEEDYESALALANGANNLYTFQLHRNSDQLHHPNLHHHRGSSLGSSITHLHDLRLA

SEQ ID NO: 16 - protein, 234 aa, Pyrus bretschneideri
MGRGKIEIKLIENQTNRQVTYSKRRNGIFKKAQELTVLCDAKVSLIMLSNTNKMHEYISPTTTTKSMYDDYQKTMGIDLWRTHDESMKDTLWKLKEINNKLRREIRQRLGHDLNGLRFDELASLDDEMQSSLDAIRQRKYHVIKTQTETTKKKVKNLEQRRGNMLHGYFDQEAASEDPQYGYEDNEGDYESALALANGANNLYTFHLHRNSDHLHHGGSSLGSSITHLHDLRLA

SEQ ID NO: 17 - protein, 237 aa, Pyrus communis
MGRGKIEIKLIENQTNRQVTYSKRRNGIFKKAQELTVLCDAKVSLIMLSNTSKMHEYISPTTTTKRMYDDYQKTMGVDLWRTHYESMKDTLWKLKEINNKLRREIRQRLGHDLNGLSYDDLRSLEDKMQSSLDAIRERKYHVIKTQTETTKKKVKNLEERRGNMLHGYEAASENPQYCYVDNEEDYESALALANGANNLYTFQLHRNSDQLHHPNLHHHRGSSLGSSITHLHDLRLA

SEQ ID NO: 18 - protein, 234 aa, Pyrus communis
MGRGKIEIKLIENQTNRQVTYSKRRNGIFKKAQELTVLCDAKVSLIMLSNTNKMHEYISPTTTTKSMYDDYQKTMGIDLWRTHHESMKDTLWKLKEINNKLRREIRQRLGHDLNGLRFDELASLDDEMQSSLDAIRQRKYHVIKTQTETTKKKVKNLEQRRGNMLHGYFDQEAASEDPQYGYEDNEGDYESALALANGANNLYTFHLHRNSDHLHHGGSSLGSSITHLHDLRLA

SEQ ID NO: 19 - DNA, 1043 bp, Malus domestica
atatcaactaaaacaagatctgaaaattactagaaaaaagtaagaaaattgagagagagattatgggtcgtggaaagattgagatcaagctgatcgaaaaccagaccaacaggcaggtgacctactccaagagaagaaatgggatcttcaagaaggctcaggagctcaccgttctctgtgatgccaaggtctccctcatcatgctctccaacactagtaaaatgcacgagtatatcagccctaccactacgaccaagagtatgtatgatgactatcagaaaactatggggatcgatctatggaggacacactacgagtctatgaaagacaccttgtggaagttgaaagagatcaacaataagctgaggagagagatcaggcagaggttgggccatgatctaaatggtctgagctatgacgatctccgttctcttgaggataagatgcaatcttccttagatgccatacgtgaaagaaagtaccatgtgatcaaaactcaaacggagaccaccaagaagaaggttaagaacttggaggaaagaagaggaaacatgctgcatggctatgaagctgccagtgagaatccacaatattgttatgtggacaatgagggagactatgaatctgcacttgtgttggcaaatggggcaaataacttgtacactttccagctccaccgcaactccgaccagctccaccaccctaacctccaccaccacagaggaagttcgctcggctcctccatcactcatctacatgatctccgccttgcttgatcgtgatccgagagatgattaatcgccactaagttttatattaaggtcacttataattttatatatattcctcagagacttaactgcttatgttctaaagtgtttttttagtgatgatctttaggcacttggattacctctaaaaacatatgcataaatatatctgggatgttttatttaatgataacactaacacaagaatctatggcctgaaagtaataatattatgaacacttccgtgcccaaaaaaaaaaaaaaaaaaaaaaa

SEQ ID NO: 20 - DNA, 1102 bp, Malus domestica
atatatcaagtaaaacaagatcagaaaattgctaggaaaaggtaagaaatttgagagagagagagaaattatgggtcgtgggaagattgaaatcaagctgatcgaaaaccagaccaacaggcaggtgacctactccaagagaagaaatgggatcttcaagaaggctcaggagctcaccgttctctgtgatgccaaggtctccctcattatgctctccaacactaataaaatgcacgagtatatcagccctaccactacgaccaagagtatgtatgatgactatcagaaaactatggggatcgatctgtggaggacacacgaggagtcgatgaaagacaccttgtggaagttgaaagagatcaacaataagctgaggagagagatcaggcagaggttgggccatgatctaaatggcctgagctttgacgagctggcttctcttgacgatgagatgcagtcttccttggatgccatacgtcaaaggaagtaccatgtgatcaaaactcagacggagaccaccaagaagaaggttaagaacttggagcaaagaagaggaaacatgctgcatggctattttgaccaggaagcagccggcgaggatccacagtatggttatgaggacaatgagggagactacgaatctgcacttgcattgtcaaatggggcgaataacttgtacactttccacctccaccaccgtaacctccaccacggaggaagttcgctcggctcctccattactcatctgcacgatctccgccttgcttgatcgtgatctgagatatgattaatcatcactaagttatatattaaggtcacttataactgcttttgttctaaagtgtttgcttggtgactatctttaggcaaggagttagacttggactacctctgaaaacagatgcataaatatgtgtgtggtgttttaatcaatgatagcactaaaaaaatccgcgcccttgttgcttgtgggtttgtttgtataattaatacttctattctatatatatcatggcagacattgcttttgatattttggtttgtttgtcttggatgtcttgtattggctttagggacttgtttggaaaagacaataactgcc

SEQ ID NO: 21 - DNA, 723 bp, Pyrus bretschneideri
atgggtcgtggaaagattgagatcaagctgatcgaaaaccagaccaacaggcaggtgacctactccaagagaagaaatgggatcttcaagaaggctcaggagctcaccgttctctgtgatgccaaggtctccctcatcatgctctccaacactagtaaaatgcacgagtatatcagccctaccactacgaccaagaggatgtacgacgactatcagaaaactatgggggtcgatctatggaggacacactacgagtcgatgaaagacaccttgtggaagttgaaagagatcaacaataagctgaggagagagatcaggcagaggttgggccatgatctaaatggtctgagctatgacgatctccgttctcttgaggataagatgcaatcttccttagatgccatacgtgaaagaaagtaccatgtgatcaaaactcagacggagaccaccaagaagaaggttaagaacttggaggaaagaagaggaaacatgctgcatggctattttgaccaggaagctgccagtgagaatccacaatattgttatgtggacaatgaggaagactatgaatctgcacttgcgttggcaaatggggcaaataacttgtacactttccagctccaccgcaactccgaccagctccaccaccctaacctccaccaccacagaggaagttcgctcggctcctccatcactcatctacatgatctccgccttgcttga

SEQ ID NO: 22 - DNA, 705 bp, Pyrus bretschneideri
atgggtcgtgggaagattgaaatcaagctgatcgaaaaccagaccaacaggcaggtgacctactccaagagaagaaatgggatcttcaagaaggctcaggagctcaccgttctctgtgatgcgaaggtctccctcattatgctctccaacactaataaaatgcacgagtatatcagccctaccactacgaccaagagtatgtatgatgactatcagaaaactatggggatcgatctgtggaggacacacgacgagtcgatgaaagacaccttgtggaagttgaaagagatcaacaataagctgaggagagagatcaggcagaggttgggccatgatctaaatggcctgcgctttgacgagctggcttctcttgacgatgagatgcagtcttccttggatgccatacgtcaaaggaagtaccatgtgatcaaaactcagacggagaccaccaagaagaaggttaagaacttggagcaaagaagaggaaacatgctgcatggctattttgaccaggaagcagccagtgaggatccacagtatggttatgaggacaatgagggagactacgaatctgcacttgcattggcaaatggggcaaataacttgtacactttccacctccaccgcaactccgaccacctccaccacggaggaagttcgctcggctcctccattactcatctgcacgatctacgccttgcttga

SEQ ID NO: 23 - DNA, 714 bp, Pyrus communis
atgggtcgtggaaagattgagatcaagctgatcgaaaaccagaccaacaggcaggtgacctactccaagagaagaaatgggatcttcaagaaggctcaggagctcaccgttctctgtgatgccaaggtctccctcatcatgctctccaacactagtaaaatgcacgagtatatcagccctaccactacgaccaagaggatgtacgacgactatcagaaaactatgggggtcgatctatggaggacacactacgagtcgatgaaagataccttgtggaagttgaaagagatcaacaataagctgaggagagagatcaggcagaggttgggccatgatctaaatggtctgagctatgacgatctccgttctcttgaggataagatgcaatcttccttagatgccatacgtgaaagaaagtaccatgtgatcaaaactcagacggagaccaccaagaagaaggttaagaacttggaggaaagaagaggaaacatgctgcatggctatgaagctgccagtgagaatccacaatattgttatgtggacaatgaggaagactatgaatctgcacttgcgttggcaaatggggcaaataacttgtacactttccagctccaccgcaactccgaccagctccaccaccctaacctccaccaccacagaggaagttcgctcggctcctccatcactcatctacatgatctccgccttgcttga

SEQ ID NO: 24 - DNA, 705 bp, Pyrus communis
atgggtcgtgggaagattgaaatcaagctaatcgaaaaccagaccaacaggcaggtgacctactccaagagaagaaatgggatcttcaagaaggctcaggagctcaccgttctctgtgatgccaaggtctccctcattatgctctccaacactaataaaatgcacgagtatatcagccctaccactacgaccaagagtatgtatgatgactatcagaaaactatggggatcgatctgtggaggacacaccacgagtcgatgaaagacaccttgtggaagttgaaagagatcaacaataagctgaggagagagatcaggcagaggttgggccatgatctaaacggcctgcgctttgacgagctggcttctcttgacgatgagatgcagtcttccttggatgccatacgtcaaaggaagtaccatgtgatcaaaactcagacggagaccaccaagaagaaggttaagaacttggagcaaagaagaggaaacatgctgcatggctattttgaccaggaagcagccagtgaggatccacagtatggttatgaggacaatgagggagactacgaatctgcacttgcattggcaaatggggcaaataacttgtacactttccacctccaccgcaactccgaccacctccaccacggaggaagttcgctcggctcctccattactcatctgcacgatctacgccttgcttga

SEQ ID NO: 25 - DNA, 430 bp, Artificial Sequence
  Construct
atggcctatgaaagcaaatccttgtccttggactctccccagagaaaattgggtaggggaaagatcgagattaagcggatcgaaaacacaacgaatcgtcaagtgaccttctgcaagaggcgcaatgggttgctcaagaaggcctatgaactctctgtgctctgtgatgcagaggttgctctcatagtcttctctaaccgtggccgcctctatgagtatgccaacaatagtgttaaaggaacaattgagaggtacaagaaggcaagtgcagattcttcaaatactggatcagtttctgaagctagtactcagtactaccagcaagaagctgcgaaattgcgtgcgcagatagtgaaattgcagaatgacaaccggaatatgatgggtgatgcattgagtagtatgtctgtcaaggacctgaagagcctgg

SEQ ID NO: 26 - DNA, 21 bp, Artificial Sequence
  Primer
atggcctatgaaagcaaatcc

SEQ ID NO: 27 - DNA, 20 bp, Artificial Sequence
  Primer
ccaggctcttcaggtccttg

SEQ ID NO: 28 - DNA, 414 bp, Artificial Sequence
  Construct
atatatcaagtaaaacaagatcagaaaattgctaggaaaaggtaagaaatttgagagagagagagaaattatgggtcgtgggaagattgaaatcaagctgatcgaaaaccagaccaacaggcaggtgacctactccaagagaagaaatgggatcttcaagaaggctcaggagctcaccgttctctgtgatgccaaggtctccctcattatgctctccaacactaataaaatgcacgagtatatcagccctaccactacgaccaagagtatgtatgatgactatcagaaaactatggggatcgatctgtggaggacacacgaggagtcgatgaaagacaccttgtggaagttgaaagagatcaacaataagctgaggagagagatcaggcagaggttgggccatgatctaaatgg

SEQ ID NO: 29 - DNA, 414 bp, Artificial Sequence
  Construct
atgggacgtgggaaggttgagatcaagaggattgagaactcaagtaacaggcaggtgacctactccaagaggaggaatgggattatcaagaaggcaaaggagatcactgttctatgtgatgctaaagtatctcttatcatttattctagctctgggaagatggttgaatactgcagcccttcaactacgctgacagaaatcttggacaaataccatggacaatctgggaagaagttgtgggatgctaagcatgagaacctcagcaatgaagtggatagagtcaagaaagacaatgacagcatgcaagtagagctcaggcatctgaagggagaggatatcacatcattgaaccatgtagagctgatggccttagaggaagcacttgaaaatggccttacaagtatccgggacaag

SEQ ID NO: 30 - protein, 245 aa, Malus domestica
MAYESKSLSLDSPQRKLGRGKIEIKRIENTTNRQVTFCKRRNGLLKKAYELSVLCDAEVALIVFSNRGRLYEYANNSVKGTIERYKKASADSSNTGSVSEASTQYYQQEAAKLRARIVKLQNDNRNMMGDALNSMSVKDLKSLENKLEKAISRIRSKKNELLFAEIEYMQKRELDLHNNNQLLRAKIAENERASRTLNVMAGGGTSSYDILQSQPYDSRNYFQVNALQPNHQYNPRHDQISLQLV

SEQ ID NO: 31 - DNA, 1037 bp, Malus domestica
ttctttattttctgcatatctccttgtttagatttgtggagctgtgaaaaaatcccaaaagcttccaactatggcctatgaaagcaaatccttgtccttggactctccccagagaaaattgggtaggggaaagatcgagattaagcggatcgaaaacacaacgaatcgtcaagtgaccttctgcaagaggcgcaatgggttgctcaagaaggcctatgaactctctgtgctctgtgatgcagaggttgctctcatagtcttctctaaccgtggccgcctctatgagtatgccaacaatagtgttaaaggaacaattgagaggtacaagaaggcaagtgcagattcttcaaatactggatcagtttctgaagctagtactcagtactaccagcaagaagctgcgaaattgcgtgcgcggatagtgaaattgcagaatgacaaccggaatatgatgggtgatgcattgaatagtatgtctgtcaaggacctgaagagcctggagaataaactggagaaagcaattagcagaatccgatccaaaaagaatgagctcttgtttgccgaaattgagtacatgcagaaaagggaactggacttgcacaacaataaccagctcctacgagcaaagatagctgagaatgagagggccagcagaacattaaatgtgatggccggaggaggaacttcaagctatgacatcctgcagtctcagccatacgactctcggaactatttccaagtgaacgcgttacaacccaatcatcagtacaatccccgccacgatcagatttctcttcaattagtatgaatgatccagattgcttggggaatgaagaccgttaccaagtacgtaggtatacgtacgtatattagttgatggaaggggatttcttgtctgtatttttatatttatgcctcctgcaacgtgtactactaagacttctgtgagacattaccctaacaagttcttccaactttgatgtgattgaatcttcataatgttggtttgaatatctattatatatgtgtctgataaa

SEQ ID NO: 32 - protein, 252 aa, Arabidopsis thaliana
TAYQSELGGDSSPLRKSGRGKIEIKRIENTTNRQVTFCKRRNGLLKKAYELSVLCDAEVALIVFSSRGRLYEYSNNSVKGTIERYKKAISDNSNTGSVAEINAQYYQQESAKLRQQIISIQNSNRQLMGETIGSMSPKELRNLEGRLERSITRIRSKKNELLFSEIDYMQKREVDLHNDNQILRAKIAENERNNPSISLMPGGSNYEQLMPPPQTQSQPFDSRNYFQVAALQPNNHHYSSAGRQDQTALQLV

SEQ ID NO: 33 - DNA, 1614 bp, Arabidopsis thaliana
ctaaatgtactgaaaagaaacaccagtttaattaattatacttccctcatatataactatcaaccaagtacaaaacttttgtcaattctcaaaatcaactttcaccacataattatctaacatgtgtatgttccaaaaccagtttaaatagaattacttttcagaaaatacatgtatattaactctatctaataaagaagaaacacatacttatctcatagattccattcataaaactatgctttagtgagtaagaaaaccagtaatcaaacacaaattgacaagacactatatggatgtaaaaagtggggaaaaatggtgataaatagtagagaaaattaaaaagaaaaaaaatattcctttataaatgtatatacccatctcttcaccagcacaaccttaccttccattttctgcaacttctccaaatctcatactttccagaaaatcattttcccaagaaaaataaaactttcccctttgttcttctccccccaacagcaatcacggcgtaccaatcggagctaggaggagattcctctcccttgaggaaatctgggagaggaaagatcgaaatcaaacggatcgagaacacaacgaatcgtcaagtcactttttgcaaacgtagaaatggtttgctcaagaaagcttacgagctctctgttctttgtgatgctgaagtcgcactcatcgtcttctctagccgtggtcgtctctatgagtactctaacaacaggtttcgtattagaatcacttaactgtgcaagtggtcgatttgaccctatcaattttattttttattacttatcaaaatgcagatttaagcggactattgagaggtacaagaaggcaatatcggacaattctaacaccggatcggtggcagaaattaatgcacagtattatcaacaagaatcagccaaattgcgtcaacaaataatcagcatacaaaactccaacaggcaattgatgggtgagacgatagggtcaatgtctcccaaagagctcaggaacttggaaggcagattagagagaagtattacccgaatccgatccaagaagaatgagctcttattttctgaaatcgactacatgcagaaaagagaagttgatttgcataacgataaccagattcttcgtgcaaagatagctgaaaatgagaggaacaatccgagtataagtctaatgccaggaggatctaactacgagcagcttatgccaccacctcaaacgcaatctcaaccgtttgattcacggaattatttccaagtcgcggcattgcaacctaacaatcaccattactcatccgcgggtcgccaagaccaaaccgctctccagttagtgtaatataggctgaaggaaatggccgggagtgaataaaaaccagaattgggttgagcaagcaatataaagctaatgcatgttatatatatatttatcccatgaatgttgtatcagtgaattttatgcttatgttgatgtgaaattaatatcttaaagacatgtcattaatgtgcttaatttgcttcaaaacatctatgtgtataagtgtactattagttcaattgttgtatttaattaccaattcctgctctattaatgattttattatag